(19) United States
(12) Patent Application Publication
(10) Pub. No.: US 2008/0221205 A1
(43) Pub. Date: Sep. 11, 2008

(54) FURAN DERIVATIVES FOR PREVENTING AND CURING OSTEOPOROSIS AND PHARMACEUTICAL COMPOSITIONS CONTAINING THE SAME

(76) Inventors: Jung-Keun Kim, Kyunggi-do (KR); Se-Won Kim, Chungcheongnam-do (KR); Kwi-Ok Oh, Kyunggi-do (KR); Seon Yle Ko, Daejeon-si (KR); Jong Yeo Kim, Chungcheongnam-do (KR); Byung-Eui Lee, Daejeon-si (KR); Bum Tae Kim, Daejeon-si (KR); Yeon Soo Lee, Daejeon-si (KR); Yong Ki Min, Daejeon-si (KR); No Kyun Park, Daejeon-si (KR)

Correspondence Address: JHK Law, P. O. Box 1078, La Canada, CA, 91012-1078, US

(21) Appl. No.: 12/125,841

(22) Filed: May 22, 2008

(30) Foreign Application Priority Data
Oct. 22, 2002 (KR) 10-2002-0064670
Oct. 17, 2003 (KR) 10-2003-0072536

(51) Int. Cl. A61K 31/341; A61P 19/10

(52) U.S. Cl. 514/461

(57) ABSTRACT

The present invention relates to furan derivatives and pharmaceutical compositions containing them to prevent and cure osteoporosis. The furan derivatives of the present invention have effect on bone proliferation with the side effect reduced, so that they can be used for bone disease.

### TECHNICAL FIELD

The present invention relates to furan derivatives and pharmaceutical compositions containing the same, more particularly, to furan derivatives having an improved effect on bone proliferation, compared to the conventional agents for treatment of osteoporosis, their pharmaceutically acceptable salts, and pharmaceutical compositions comprising such furan derivatives as effective ingredients.

### BACKGROUND ART OF THE INVENTION

Osteoporosis is a disease caused by reduction of the content of calcium which determines the physical strength of bone by various reasons (genetic factors, insufficient intake of nutrients, change of hormones, physical inactivity, habits, etc.). In this disease, the medullary cavity is enlarged, and bone becomes easily fractured even by a weak impact, resulting in tremendous inconvenience. In particular, in case of women, bone mineral density starts to decrease gradually after age 30, and estrogen levels drop sharply during menopause. During estrogen deficiency, the production of B lymphocytes is increased, such as by action of interleukin 7 (IL-7), and B cell progenitors are thus accumulated in the bone marrow, thereby promoting IL-6 synthesis. Eventually, the cytokine IL-6 stimulates osteoclasts, and bone mineral density is thus reduced.

The currently available therapeutic agents for osteoporosis include bisphosphonates (e.g., alendronate, etidronate), hormones (e.g., raloxifene), vitamin D and its analogues, calcitonin and calcium. However, bisphosphonates are disadvantageous in terms of being slowly absorbed by the gastrointestinal tract, being administered by an intricate procedure, and causing inflammation in the esophagus when administration is not successfully achieved. The hormonal drugs should be administered for the whole life, and increase the prevalence of breast cancer and uterine cancer. The vitamin preparations are expensive and not clearly identified for its therapeutic efficacy for osteoporosis. Also, calcitonin is expensive and its administration is difficult. The calcium preparations are known to have mild side effects, but are a preventive, not therapeutic, agent.

In detail, the conventional osteoporosis therapeutic agents have significant disadvantages (Medical Information Newsletters, Vol. 24, No. 10, 1998), as follows. Bisphosphonates, which was approved from the FDA of U.S.A 1995 November, inhibit strongly bone absorption and has an excellent effect on treating osteoporosis in postmenopausal women. However, they should be administered 30 min to 1 hr before a meal and has a low absorption rate by the body. Calcitonin is effective in bone absorption and relieves the pain associated with the bone fractures by acting to the hypothalamus. However, calcitonin has a weak therapeutic effect on osteoporosis when used for a long period and is relatively expensive compared to its therapeutic effect, and its administration is complex. Sex hormones are mainly used for prevention, rather than treatment, of menopausal disorders and cardiovascular diseases, and effective in treating osteoporosis by inhibiting osteoclasts. However, sex hormones have side effects including the risk of breast cancer and bleeding, and are not convenient in use. Also, the calcium preparations aid only skeleton formation in rapidly growing children, juveniles and pregnant or lactating women.

### SUMMARY OF THE INVENTION

Leading to the present invention, the thorough and intensive research with aiming to improve the problematic conventional osteoporosis therapeutic agents, conducted by the present inventors, resulted in the finding that furan derivatives have a better effect on suppression of bone proliferation and have milder side effects, compared to the conventional therapeutic agents for osteoporosis.

It is an object of the present invention to provide furan derivatives that are effective in preventing and treating osteoporosis, and their pharmaceutically acceptable salts.

It is another object of the present invention to provide pharmaceutical compositions for preventing and treating osteoporosis, comprising such furan derivatives as effective ingredients.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing degree of proliferation of MG63 osteoblasts when treated with the Compound 1 of the present invention, prepared in Example 1, at various concentrations, in comparison with a control not treated with the compound;

FIG. 2 is a graph showing alkaline phosphatase (ALP) activity when HOS cells were treated with the Compound 1 of the present invention, prepared in Example 1, in comparison with a control not treated with the compound;

FIG. 3 is a graph showing expression levels of Runx2 that is a transcription factor for differentiation of osteoblasts when 6xOSE2-Luc vector-transfected C2C12 cells were treated with the Compound 1 of the present invention, prepared in Example 1, in comparison with a control not treated with the compound;

FIG. 4 is a graph showing expression levels of osteoprotegerin (OPG) when MG63 osteoblasts were treated with the Compound 1 of the present invention, prepared in Example 1, where the OPG expression levels were analyzed by an ELISA assay;

FIG. 5 is a graph showing production of TRAP (tartrate-resistant acid phosphatase)-positive multinucleated cells when osteoclast progenitor cells were treated with the Compound 1 of the present invention, prepared in Example 1;

FIG. 6 is a graph showing the area of formed resorption pits when osteoclast progenitor cells, cultured in calcium phosphate-coated plates, were treated with the Compound 1 of the present invention, prepared in Example 1, in comparison with a control not treated with the compound;

FIGS. 7a and 7b are microscopic photographs (12.5× magnification) showing no trabecular bone loss in ovariectomized white mice orally administered for four weeks with 10 mg/day of the Compound 1 of the present invention, prepared in Example 1, where 7a and 7b show cross-sectional views of leg bone of white mice not treated and treated with the Compound 1, respectively;

FIGS. 8a and 8b are microscopic photographs (40× magnification) showing no trabecular bone loss in ovariectomized white mice orally administered for four weeks with 10 mg/day of the Compound 1 of the present invention, prepared in Example 1, where 8a and 8b show cross-sectional views of leg bone of white mice not treated and treated with the Compound 1, respectively;

FIGS. 9a and 9b are microscopic photographs (40× magnification) showing no trabecular bone loss in ovariectomized white mice subcutaneously administered for four weeks with 10 mg of the Compound 1 of the present invention, prepared in Example 1, where 9a and 9b show cross-sectional views of leg bone of white mice not treated and treated with the Compound 1, respectively;

FIGS. 10a and 10b are microscopic photographs (40× magnification) showing no trabecular bone loss in ovariectomized white mice bred with solid foodstuff for four weeks to develop osteoporosis and then orally administered for four weeks with 220 μl/day of the Compound 1 of the present invention, prepared in Example 1, where 10a and 10b show cross-sectional views of leg bone of white mice not treated and treated with the Compound 1, respectively;

FIG. 11 is a graph showing remarkably increased bone mineral density (BMD) in ovariectomized white mice orally administered for four weeks with 10 mg/day of the Compound 1 of the present invention, prepared in Example 1, where, four weeks after ovariectomy, BMD was elevated compared to that before ovariectomy; and

FIG. 12 is a graph showing a very slight reduction of BMD in ovariectomized white mice bred with solid foodstuff for four weeks to develop osteoporosis and then orally administered for four weeks with 220 μl/day of the Compound 1 of the present invention, prepared in Example 1, compared to a control.

### DETAILED DESCRIPTION OF THE INVENTION

The present invention provides a furan derivative represented by the following Formula 1, and their pharmaceutically acceptable salts:

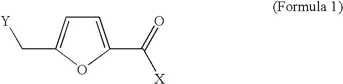

wherein, X represents H, OH, OR or NR¹R² and Y represents OR, NR¹R² or SC(═NH₂)NH; and

wherein, R represents hydrogen, naphthalene, aryl group having three or less substitution groups selected from among methyl, methoxy, chloro, bromo, iodo, nitro and fluorine, or a C₁-C₄ aliphatic alkyl group having four or less substituted fluorine; and

R¹ and R² are the same or different from each other and each represents hydrogen, naphthalene, aryl group having three or less substitution groups selected from among methyl, methoxy, chloro, bromo, iodo, nitro and fluorine, or a C₁-C₃ aliphatic alkyl group, or R¹ and R² are linked with carbon, oxygen, hydrogen, or nitrogen having an C₁-C₃ aliphatic alkyl group and together represent an aliphatic alkyl group.

In detail, illustrative examples of the furan derivative represented by the Formula I include furan derivatives having substitution groups, X and Y, listed in Tables 1 to 7, below.

TABLE 1

| No. | X | Y |
|---|---|---|
| 1 | H | HO— |
| 2 | H | CH₃COO— |
| 3 | H | C₆F₅O— |
| 4 | H | CH₃O— |
| 5 | H | 3,4-Cl₂C₆H₃O— |
| 6 | H | 4-NO₂C₆H₄O— |
| 7 | H | 2,4,6-Cl₃C₆H₂O— |
| 8 | H | 4-BrC₆H₄O— |
| 9 | H | 3-CH₃-4ClC₆H₃O— |
| 10 | H | C₆Cl₅O— |
| 11 | H | 4-CNC₆H₄O— |
| 12 | H | 3-CF₃C₆H₄O— |
| 13 | H | 4-CH₃OC₆H₄O— |
| 14 | H | 2,4-F₂C₆H₃O— |
| 15 | H | 3-BrC₆H₄O— |
| 16 | H | 2-NO₂C₆H₄O— |
| 17 | H | 2-BrC₆H₄O— |
| 18 | H | 3-Cl-4-FC₆H₃O— |
| 19 | H | 2-Cl-4-BrC₆H₃O— |
| 20 | H | 2-CH₃OC₆H₄O— |
| 21 | H | 3-CH₃-4-NO₂C₆H₃O— |
| 22 | H | 2-Cl-4-FC₆H₃O— |
| 23 | H | 2,3-Cl₂C₆H₃O— |
| 24 | H | 2-NO₂-4-ClC₆H₃O— |
| 25 | H | 4-ClC₆H₄O— |
| 26 | H | 2,4-Cl₂C₆H₃O— |
| 27 | H | 2-(CH₃)₂CH-4-Cl-5-CH₃C₆H₂O— |
| 28 | H | 2,4,6-Br₃C₆H₂O— |
| 29 | H | 2-CH₃C₆H₄O— |
| 30 | H | 2,6-(CH₃)₂C₆H₃O— |

TABLE 2

| No. | X | Y |
|---|---|---|
| 31 | H | C₆H₅COO— |
| 32 | H | C₆H₅CH₂COO— |
| 33 | H | 2,6-F₂C₆H₃CH₂COO— |
| 34 | H | 2-Cl-6-FC₆H₃CH₂COO— |
| 35 | H | 3-Cl—C₆H₄CH₂COO— |
| 36 | H | 3-SC₄H₃CH₂COO— |
| 37 | H | 3-F—C₆H₄CH₂COO— |
| 38 | H | 2-NpCH₂COO— |
| 39 | H | 2,4-F₂C₆H₃CH₂COO— |
| 40 | H | (C₆H₅)₃CCOO— |
| 41 | H | 2-CH₃O-6-FC₆H₃CH₂COO— |
| 42 | H | 3-CH₃O-6-FC₆H₃CH₂COO— |
| 43 | H | 2-BrC₁₄H₂₈COO— |
| 44 | H | C₁₄H₂₉COO— |
| 45 | H | 4-FC₆H₄NHCOO— |
| 46 | H | C₆H₅NHCOO— |
| 47 | H | (CH₃)₂CHNHCOO— |
| 48 | H | 3-CF₃C₆H₄NHCOO— |
| 49 | H | 3-ClC₆H₄NHCOO— |
| 50 | H | 4-BrC₆H₄NHCOO— |
| 51 | H | 2,4-(CH₃O)₂C₆H₃NHCOO— |
| 52 | H | C₆H₁₁NHCOO— |
| 53 | H | CH₃CH₂CH₂CH₂NHCOO— |
| 54 | H | 3,4-Cl₂C₆H₃NHCOO— |
| 55 | H | 2-ClC₆H₄NHCOO— |
| 56 | H | CH₃CH₂NHCOO— |
| 57 | H | 2-NpNHCOO— |
| 58 | CH₃O— | 3,5-Cl₂-4-NH₂C₆H₂C (NH₂)═NO— |
| 59 | CH₃O— | 2-CH₃O-4-CH₂═CHCH₂C₆H₃O— |
| 60 | CH₃O— | 2,4-Cl₂C₆H₄O— |

TABLE 3

| No. | X | Y |
|---|---|---|
| 61 | CH₃O— | 2-ClC₆H₄O— |
| 62 | CH₃O— | 2-BrC₆H₄O— |
| 63 | CH₃O— | 2,4-Cl₂C₆H₄O— |
| 64 | CH₃O— | 2-NpO—* |
| 65 | CH₃O— | C₆F₅O— |
| 66 | CH₃O— | 2-NO₂-4-ClC₆H₃O— |
| 67 | CH₃O— | 2-NO₂C₆H₄O— |
| 68 | CH₃O— | 2-(CH₃)₂CH-5-CH₃C₆H₃O— |
| 69 | CH₃O— | 4-Cl—C₆H₄O— |
| 70 | CH₃O— | 3,4-(CH₂)₃C₆H₃O— |
| 71 | CH₃O— | 2-Cl-4-BrC₆H₃O— |
| 72 | CH₃O— | 2-Cl-4-FC₆H₃O— |
| 73 | CH₃O— | 3-CH₃C₆H₄O— |
| 74 | CH₃O— | 2-CH₃-4-ClC₆H₃O— |
| 75 | CH₃O— | 3-CH₃-4-ClC₆H₃O— |
| 76 | CH₃O— | 2,4-(CH₃)C₆H₃O— |
| 77 | CH₃O— | 3,5-(CH₃)₂-4-ClC₆H₂O— |
| 78 | CH₃O— | 4-(CH₃)₂CHC₆H₄O— |
| 79 | CH₃O— | 4-IC₆H₄O— |
| 80 | CH₃O— | 4-ClC₆H₄O— |
| 81 | CH₃O— | 3,4-(CH₃)₂C₆H₃O— |
| 82 | CH₃O— | HN═C(NH₂)S— |
| 83 | CH₃O— | 2-NpO—* |
| 84 | CH₃O— | C₆F₅O— |
| 85 | CH₃O— | (CH₃)₂N— |
| 86 | CH₃O— | HN═C(NH₂)S— |
| 87 | CH₃O— | (CH₂)₅N— |
| 88 | CH₃O— | O(CH₂CH₂)₂N— |
| 89 | CH₃O— | C₆H₅NH— |
| 90 | CH₃O— | (CH₂)₄N— |

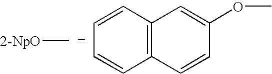

TABLE 4

| No. | X | Y |
|---|---|---|
| 91 | CH₃O— | (CH₃)₃CNH— |
| 92 | CF₃CH₂O— | 2-NpO—* |
| 93 | (CH₃)₂CHO— | 4-(CH₃)₂CHC₆H₄O— |
| 94 | (CH₃)₂CHO— | 2-CH₃OC₆H₄O— |
| 95 | (CH₃)₂CHO— | 2,5-Cl₂C₆H₃O— |
| 96 | 2-ClC₆H₄O— | CH₃CH₂OC₆H₄O— |
| 97 | 4-ClC₆H₄O— | 2-CH₃OC₆H₄O— |
| 98 | C₆H₅O— | 2-ClC₆H₄O— |
| 99 | CH₂═CHCH₂O— | 2,4-Cl₂C₆H₃O— |
| 100 | HO— | 4-CH₃CH₂CH₂C₆H₄O— |
| 101 | HO— | CHF₂CF₂CH₂O— |
| 102 | HO— | 4-FC₆H₄O— |
| 103 | HO— | 4-BrC₆H₄O— |
| 104 | HO— | 2-NpO—* |
| 105 | HO— | 3-CF₃C₆H₄C(CH₃)═NO— |
| 106 | HO— | 2,4-Cl₂C₆H₃O— |
| 107 | HO— | 2-ClC₆H₄O— |
| 108 | HO— | 2-BrC₆H₄O— |
| 109 | HO— | 2,5-Cl₂C₆H₃O— |
| 110 | HO— | 4-FC₆H₄O— |
| 111 | HO— | 4-Cl-3-CH₃C₆H₄O— |
| 112 | HO— | 3-ClC₆H₄O— |
| 113 | HO— | 2-NO₂C₆H₄O— |
| 114 | HO— | 4-(CH₃)₂CHC₆H₄O— |
| 115 | HO— | 4-Cl-2-NO₂C₆H₃O— |
| 116 | HO— | 3-CH₃OC₆H₄O— |
| 117 | HO— | 1-NpO— |
| 118 | HO— | 4-CH₃CH₂CH(CH₃)C₆H₄O— |
| 119 | HO— | 4-Cl-3-CH₃C₆H₃O— |
| 120 | HO— | C₆H₅CH₂S— |

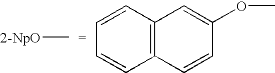

TABLE 5

| No. | X | Y |
|---|---|---|
| 121 | CH₃CH₂CH(CH₃)NH— | 4-IC₆H₄O— |
| 122 | 3-CH₃C₆H₄NH— | 4-IC₆H₄O— |
| 123 | (CH₃CH₂)₂N | 4-Cl-3-CH₃C₆H₃O— |
| 124 | CH₃CH(CH₂CH₂)₂N— | 2,5-Cl₂C₆H₄O— |
| 125 | (CH₂)₄CHNH— | CF₃CH₂O— |
| 126 | (CH₂)₆CHNH— | 2-CH₃OC₆H₄O— |
| 127 | (CH₃)₃CNH— | CF₃CH₂O— |
| 128 | (CH₂)₆N— | 2-BrC₆H₄O— |
| 129 | (CH₃)₃CNH— | 2-BrC₆H₄O— |
| 130 | (CH₃)₂CHNH— | 2,5-Cl₂C₆H₃NH— |
| 131 | CH₃N(CH₂CH₂)₂N— | 2,5-Cl₂C₆H₃O— |
| 132 | O(CH₂CH₂)₂N— | 3-CH₃-4-ClC₆H₃O— |
| 133 | (CH₂)₆N— | 2,5-Cl₂C₆H₃O— |
| 134 | (CH₂)₅CHNH— | 4-(CH₃)₃CC₆H₄O— |
| 135 | (CH₂)₄NH— | 4-IC₆H₄O— |
| 136 | C₆H₅NH— | 4-(CH₃)₃CC₆H₄O— |
| 137 | C₆H₅NH— | 2-NpO—* |
| 138 | 4-ClC₆H₄NH— | 4-IC₆H₄O— |
| 139 | 3-F-4-CH₃C₆H₃NH— | 4-IC₆H₄O— |
| 140 | 3-BrC₆H₄NH— | 4-FC₆H₄O— |
| 141 | 4-FC₆H₄NH— | 3-ClC₆H₄O— |
| 142 | 3-Cl-4-CH₃OC₆H₃NH— | 2-ClC₆H₄O— |
| 143 | 3,4-F₂C₆H₃NH— | 4-IC₆H₄O |
| 144 | 2-CH₃CH₂OC₆H₄NH— | 2-NO₂-4-ClC₆H₃O— |
| 145 | 2,4-(CH₃O)₂C₆H₃NH— | 2,5-Cl₂C₆H₃O— |
| 146 | 4-BrC₆H₄NH— | 4-IC₆H₄O— |
| 147 | 4-FC₆H₄NH— | 2-NO₂-4-CH₃C₆H₃O— |
| 148 | 4-NH₂COC₆H₄NH— | 2,5-Cl₂C₆H₃O— |
| 149 | 2-NO₂-4-CH₃OC₆H₃NH— | 4-F—C₆H₄O— |
| 150 | 4-CH₃OC₆H₄NH— | 3-CH₃-4-ClC₆H₃O— |

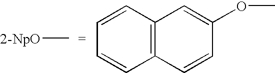

TABLE 6

| No. | X | Y |
|---|---|---|
| 151 | 2,5-F₂C₆H₃NH— | 3-ClC₆H₄O— |
| 152 | 2-CH₃-5-CH₃O₂CC₆H₃NH— | 4-IC₆H₄O— |
| 153 | 2-CH₃O₂CC₆H₄NH— | 2,5-(CH₃)₂C₆H₃O— |
| 154 | 3,5-(CH₃)₂C₆H₃NH— | 4-F—C₆H₄O— |
| 155 | 2-F-5-CH₃C₆H₃NH— | 2-NO₂-4-CH₃C₆H₃O— |
| 156 | 2,3-Cl₂C₆H₃NH— | 2-NO₂C₆H₄O— |
| 157 | 2-CH₃OC₆H₄NH— | 2,5-(CH₃)₂C₆H₃O— |
| 158 | 2-F-5-CH₃C₆H₃NH— | 2-NO₂C₆H₄O— |
| 159 | 2-CH₃O₂CC₆H₄NH— | 4-I—C₆H₄O— |
| 160 | 4-CH₃COC₆H₄NH— | 1-NpO— |
| 161 | 2,5-F₂C₆H₃NH— | 2-ClC₆H₄O— |
| 162 | 2-F-4-BrC₆H₃NH— | 4-IC₆H₄O— |
| 163 | 3-CH₃CH₂C₆H₄NH— | 4-F—C₆H₄O— |
| 164 | 3,4-(CH₃O)₂C₆H₃NH— | 4-CH₃CH₂CH₂C₆H₄O— |
| 165 | (CH₃)₃CNH— | 4-CH₃CH₂OC₆H₄O— |
| 166 | 2-CH₃OC₆H₄NH— | 2-CH₃OC₆H₄O— |
| 167 | 2-CH₃O₂CC₆H₄NH— | 2,4-Cl₂C₆H₃O— |
| 168 | 2-CH₃O₂CC₆H₄NH— | 4-ClC₆H₄O— |
| 169 | 3,5-(CH₃)₂C₆H₃NH— | 4-CH₃CH₂—C₆H₄O— |
| 170 | 2,5-Cl₂C₆H₃NH— | 4-CH₃CH₂CH₂C₆H₄O— |
| 171 | 2-CH₃O-5-CH₃C₆H₃NH— | 3-ClC₆H₄O— |
| 172 | 2,3-(CH₃)₂C₆H₃NH— | 3-CH₃-4-ClC₆H₃O— |
| 173 | 4-ClC₆H₄NH— | 3-CH₃-4-ClC₆H₄O— |
| 174 | 2-ClC₆H₄NH— | 2-CH₃OC₆H₄O— |
| 175 | 3,4-(CH₃)₂C₆H₄NH— | 2-CH₃OC₆H₄O— |
| 176 | 2,4-F₂C₆H₃NH— | 2-CH₃O-4-CH₃CH₂CH₂C₆H₃O— |
| 177 | 2-FC₆H₄NH— | 3,5-(CH₃)₂C₆H₃O— |
| 178 | 2-FC₆H₄NH— | 4-ClC₆H₄O— |
| 179 | 2,6-(CH₃)₂C₆H₃NH— | 4-ClC₆H₄O— |

TABLE 7

| No. | X | Y |
|---|---|---|
| 180 | 2-CH₃O₂CC₆H₄NH— | 3,4-(CH₃)₂C₆H₃O— |
| 181 | 2-Cl-5-CF₃C₆H₃NH— | 4-ClC₆H₄O— |
| 182 | 2-CH₃O-4-NO₂C₆H₃NH— | 4-ClC₆H₄O— |
| 183 | 2-NO₂C₆H₄NH— | 2-ClC₆H₄O— |
| 184 | 4-CH₃OC₆H₄NH— | CHF₂CF₂CH₂O— |
| 185 | 2,4-F₂C₆H₃NH— | 2,3,5,6-F₄C₆HO— |
| 186 | 3,4-F₂C₆H₃NH— | 3,4-(CH₃)₂C₆H₃O— |
| 187 | 3-CH₃CONHC₆H₄NH— | C₆F₅O— |
| 188 | 2,4-(CH₃O)₂C₆H₄NH— | 2-BrC₆H₄O— |
| 189 | 2-CH₃OC₆H₄NH— | 4-(CH₃)₃CC₆H₄O— |
| 190 | 4-IC₆H₄NH— | 2-BrC₆H₄O— |
| 191 | 3-NO₂C₆H₄NH— | 4-IC₆H₄O— |

In addition to the furan derivatives represented by the Chemical Formula I and pharmaceutically acceptable salts thereof, the present invention includes all possible solvates and hydrates prepared therefrom. The compounds of the Chemical Formula I may be used in the form of pharmaceutically acceptable salts. The salts may be prepared as pharmaceutically acceptable metal salts by using pharmaceutically acceptable bases. Alkali or alkaline earth metal salts are obtained, for example, by dissolving compounds in an excessive alkali metal hydroxide or alkali earth metal hydroxide solution, filtering the resulting solution to remove undissolved compounds, and evaporating and drying the filtrate. Herein, it is pharmaceutically suitable that the metal salts are prepared in the form of sodium, potassium or calcium salts. In addition, silver salts may be prepared by reacting alkali metal or alkali earth metal salts with a suitable silver salt (e.g., silver nitrate).

The furan derivatives of the present invention may be prepared by extraction and chemical synthesis processes commonly used in the art, but not limited to them.

In an embodiment, the furan derivatives of the present invention may be prepared, as follows.

(1) Among the compounds of the present invention, furan-2-carboxyaldehyde derivatives in which X is H, with reference to the Reaction Scheme 1, below, are prepared by reacting 5-chloromethylfuran-2-carboxyaldehyde with substituted aliphatic alcohol, substituted arylalcohol or various amines in a solvent of acetonitrile using potassium carbonate to produce 5-substituted methyl furan-2-carboxyaldehyde (when X is H and Y is OH, the furan-2-carboxyaldehyde compound was isolated from a plant Rehmannia glutinosa Libosch by an extraction process and evaluated for physiological activity). Herein, the 5-chloromethylfuran-2-carboxyaldehyde compound, used as a starting material and represented by the Formula II, is prepared from glucose and hydrochloric acid according to the method described by W. N. Haworth and W. G. M. Jones, J. Chem. Soc. 667-670, 1944.

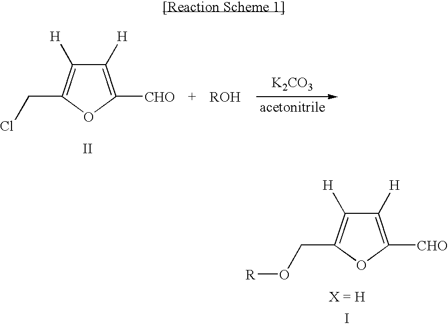

(2) Among the compounds of the present invention, furan-2-carboxyaldehyde derivatives in which X is not H, with reference to the Reaction Scheme 2, below, are obtained by primarily producing 5-chloromethylfuran-2-carboxychloride of the Formula IV using 5-hydroxymethylfuran-2-carboxylic acid of the Formula III, reacting the first product with various alcohols and amine derivatives to produce 5-chloromethyl furan-2-carboxylic acid ester and amide derivatives, and reacting the second product with a neucleophile selected from among various alcohols, amines and thioureas to produce 5-substituted methyl furan-2-carboxylic acid ester and amide derivatives of the Formula I.

Herein, the 5-hydroxymethylfuran-2-carboxylic acid, used as a starting material and represented by the Formula III, is prepared according to the method described by W. N. Haworth et al., J. Chem. Soc. 1513-1526, 1927.

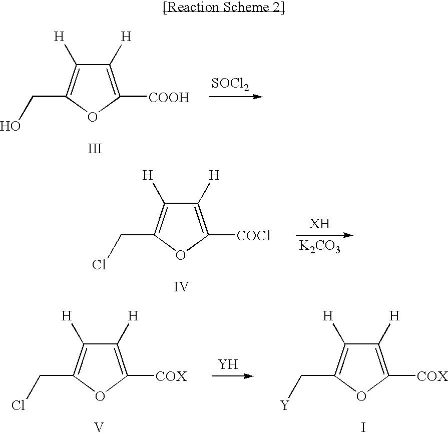

In addition, the present invention includes a pharmaceutical composition for preventing and treating osteoporosis, comprising a furan derivative represented by the Formula I or its pharmaceutically acceptable salt as an effective ingredient.

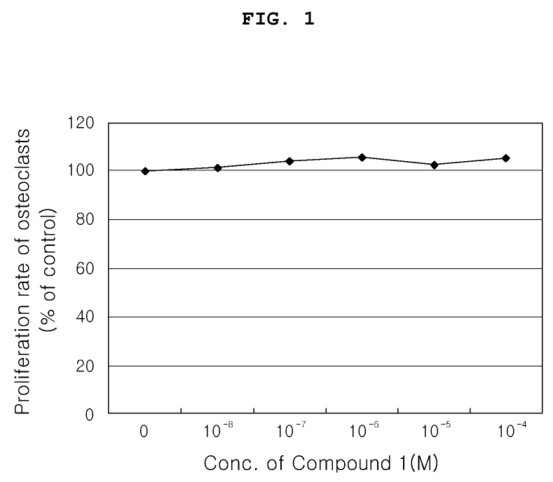

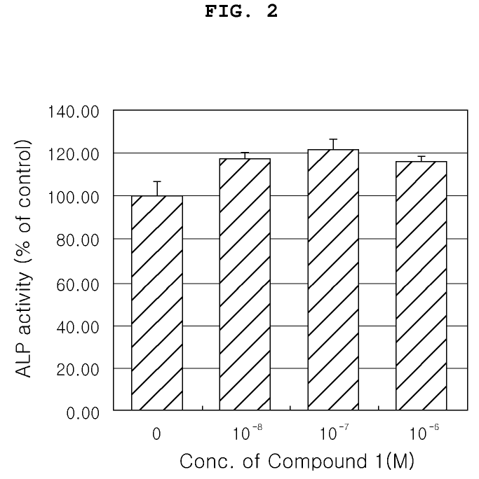

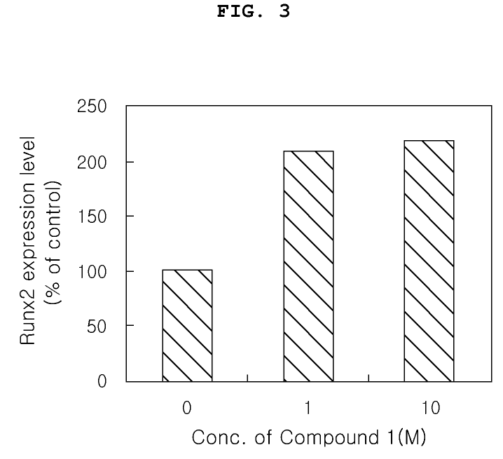

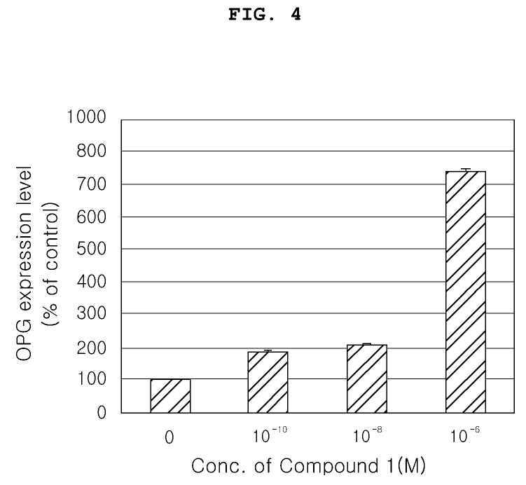

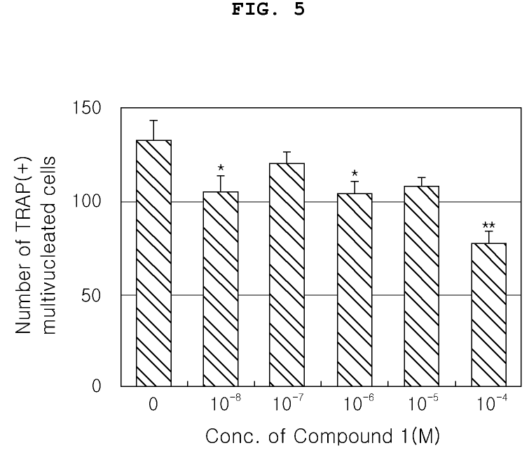

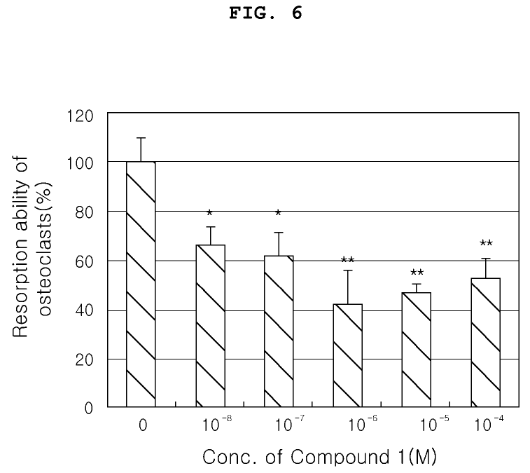

The furan derivatives of the present invention are superior to the conventional therapeutic agents for osteoporosis, by displaying effects of stimulating proliferation of osteoblasts and increasing osteoblast activity as well as inhibiting proliferation and activity of osteoclasts. In detail, as shown in Experimental Examples, below, when osteoblasts were treated with a compound prepared in Example 1, cell proliferation was increased to 105% or higher (see, FIG. 1), while phosphatase activity was elevated to 120% (see, FIG. 2), compared to a control not treated with the compound. Also, the compound prepared in Example 1 was found to increase the activity of Runx2 that is a transcription factor for differentiation of osteoblasts (see, FIG. 3). Further, the compound of the present invention, prepared in Example 1, increases the expression of osteoprotegerin (OPG) in osteoblasts, which inhibits osteoclast formation (see, FIG. 4). When TRAP-positive multinucleated cells were treated with the compound, their number was remarkably reduced (see, FIG. 5). Also, the compound was found to inhibit bone resorption activity of osteoclasts (see, FIG. 6). Clinical studies with animal models demonstrated that the compound has effects of preventing and treating osteoporosis. A pharmaceutical composition comprising the furan derivative having the aforementioned effects according to the present invention increases bone proliferation, and is thus useful for increasing children's height and preventing and treating osteoporosis, degenerative bone diseases, rheumatoid arthritis and other bone-related diseases.

Administrable via oral or parenteral routes, the compounds of formula I can be used with oral, intravenous, subcutaneous, intranasal, intrabronchial or rectal administration, and may be used with ordinary medicine forms.

That is, the compounds of formula I can be formulated into various dosage forms for oral or parenteral administration. For formulation, pharmaceutically acceptable diluents, expedients and/or carriers including fillers, thickeners, binders, wetting agent, disintegration, surfactants, etc, may be used. Solid dosage forms for oral administration are exemplified by tablets, pills, powders, granules and capsules. These solid forms are prepared by admixing at least one compound of formula I with at least one expedient such as starch, calcium carbonate, sucrose, lactose, gelatin, etc. In addition to expedients, a lubricant such as magnesium styrate talc may be added.

Suspensions, internal solutions, emulsions, syrups, etc., are liquid dosage forms for oral administration that can comprise wetting agents, sweeteners, aromatics, and/or perspectives in addition to simple diluents such as water and liquid paraffin.

Dosage forms for parenteral administration include sterile aqueous solutions, non-aqueous solvents, suspensions, emulsions, freeze-dried agents, suppositories, etc. For formulation of non-aqueous solvents and suspensions, vegetable oils such as propylene glycol and polyethylene glycol or injectable esters such as ethyl oleate may be used. As bases for suppositories, Witepsol, macrogol, Tween 61, cocoa oil, laurinic acid and glycerogelatine are useful.

Administration dosage of the compound, represented by above formula I is dependant on patient's condition, for example age, weight, sex, hygienic condition and seriousness of disease. If drug is administered to adult patient weighing 70 kg, administration dosage is generally 0.01-1000 g per 1 day, preferably 0.1-500 mg per 1 day. According to diagnosis of doctor or pharmaceutist, drug can be administrated to patient once or many times at regular intervals.

Also, the present invention provides functional foods, health-supporting food or special nutritional food, comprising of furan derivatives as effective ingredients.

The term “functional food”, as used herein, is intended to indicate a food that is made by adding the furan derivatives and pharmaceutically acceptable salts thereof to a general food to improve the functionality of the general food. The term “health-supporting food” or “special nutritional food”, as used herein separately from the functional food, refers to a health food imparting unique health benefits upon uptake, which is made by adding the furan derivatives and pharmaceutically acceptable salts thereof to a general food, or additionally by formulating the resulting general food into capsules, powders, suspensions, and the like. The health-supporting and special nutritional foods are advantageous in terms of being prepared using a food as a raw material unlike general medical drugs and thus not having side effects found in the drugs. In the functional food, health-supporting food and special nutritional food, the content of the furan derivatives and pharmaceutically acceptable salts thereof may vary depending on kinds of foods, and may be determined in the range of cytotoxicity evaluated in the use as the pharmaceutical composition.

### EXAMPLE

The present invention will be explained in more detail with reference to the following an example in conjunction with the accompanying drawings. However, the following example is provided only to illustrate the present invention, and the present invention is not limited to the example.

### Example 1

### Preparation of the Furan Derivatives of the Present Invention

(1) Preparation of 5-hydroxymethylfuran-2-carboxyaldehyde (Compound 1)

600 g of a plant Rehmannia glutinosa Libosch (steamed with ethanol (alcoholic drink: raw rice wine)) was mixed with 3 L of distilled water in an extracting reactor, and heat-extraction was carried out twice at 95° C. The extracts were put together and concentrated under pressure at below 40° C.

The concentrate was chromatographed in an open column of silica gel using ethylacetate and n-hexane as solvents, thus yielding 720 mg of 5-hydroxymethylfuran-2-carboxyaldehyde (melting point: 32-35° C.).

(2) Preparation of oxymethylfuran-2-carboxyaldehyde Having a Substitution Group (Compounds 2 to 30)

Potassium carbonate (1 mmol) was added to a mixture of 143 mg (1 mmol) of 5-chloromethylfurancarboxyaldehyde, an alcoholic compound (1-2 mmol) and acetonitrile (10 ml), and the reaction mixture was stirred for 5 hrs at room temperature. After checking completion of the reaction by thin layer chromatography, the solvent was removed from the reaction solution using a vacuum evaporator. Then, ethylacetate (30 ml) and water (30 ml) were added to the residue. The separated organic layer was dried and filtered, and subjected to column chromatography, thus giving oxymethylfuran-2-carboxyaldehyde having a substitution group, with a yield of 50-70%.

(3) Preparation of 5-chloromethylfuran-2-carboxylic Acid Methylester (V, X=—OCH₃)

Dimethylformamide (1 ml) was added to a mixture of 5-hydroxymethylfurancarboxylic acid (100 mmol), thionyl chloride (150 mmol) and toluene (100 ml), and the resulting reaction mixture was refluxed for 5 hrs. After completion of the reaction, the reaction solution was distilled at atmospheric pressure to remove the solvent and excessive thionyl chloride, thus generating 5-chloromethylfurancarbonylchloride (IV) in a liquid state. To the obtained compound, anhydrous methanol (50 ml) was added at room temperature and then powdered potassium carbonate (200 mmol) was added, followed by mixing with stirring for 1 hr. After removing methanol using a vacuum evaporator, water (100 ml) and ethylacetate (100 ml) were added to the residue to separate an organic layer. The organic layer was dried with magnesium sulfate and filtered. After removing the organic solvent from the filtrate using a vacuum evaporator, the residue was subjected to column chromatography, thus giving 5-chloromethylfuran-2-carboxylic acid methylester with a yield of 70%.

(3-1) Preparation of 5-substituted oxymethylfuran-2-carboxylic Acid Methylester (Compounds 31 to 44)

An acyl compound (1 mmol) was added to a mixture of 5-hydroxymethylfurancarboxyaldehyde (1 mmol), triethylamine (2 mmol) and acetonitrile (10 ml), and the reaction mixture was stirred for 5 hrs at room temperature. After checking completion of the reaction by thin layer chromatography, the solvent was removed from the reaction solution using a vacuum evaporator. Then, ethylacetate (10 ml) and water (10 ml) were added to the residue. The separated organic layer was dried and filtered, and subjected to column chromatography, thus giving a desired compound with a yield of 50-70%.

(3-2) Preparation of 5-aminocarbonyloxymethylfuran-2-carboxyaldehyde (Compounds 45 to 57)

5-hydroxymethyl-2-furfural (2 mmol) was dissolved in tetrahydrofuran (5 ml), and a flow of nitrogen was introduced to the mixture. An isocyanate derivative (2.2. mmol) and then a small amount of triethylamine (0.5 ml) were added to the mixture. After being stirred for 3-6 hrs at room temperature, the reaction solution was added to water (50 ml). An organic layer was separated with ethylacetate (25 ml×3), dried with magnesium sulfate anhydrous, and concentrated under pressure to remove the solvent. The resulting residue was subjected to column chromatography, thus generating a carbamate compound (yield: 95-98%).

(3-3) Preparation of 5-substituted oxymethylfuran-2-carboxylic Acid (Compounds 100 to 120)

Potassium carbonate (2 mmol) was added to a mixture of 5-chloromethylfurancarboxilic acid (1 mmol), an alcoholic or amine compound (1-2 mmol) and acetonitrile (10 ml), and the reaction mixture was stirred for 5 hrs at room temperature. After checking completion of the reaction by thin layer chromatography, the reaction solution was neutralized with 1 M hydrochloric acid, and the solvent was removed therefrom using a vacuum evaporator. Then, ethylacetate (10 ml) and water (10 ml) were added to the residue. The separated organic layer was dried and filtered, and subjected to column chromatography, thus giving a desired compound with a yield of 50-70%.

(4) Preparation of 5-chloromethylfuran-2-carboxylic Acid Ester, Amide (V, X=—OR, —NR)

Dimethylformamide (1 ml) was added to a mixture of 5-hydroxymethylfurancarboxylic acid (100 mmol), thionyl chloride (150 mmol) and toluene (100 ml), and the resulting reaction mixture was refluxed for 5 hrs. After completion of the reaction, the reaction solution was distilled at atmospheric pressure to remove the solvent and excessive thionyl chloride, thus generating 5-chloromethylfurancarbonylchloride (IV) in a liquid state. After dissolving the obtained compound in toluene (30 ml), to the resulting solution, anhydrous methanol (110 mmol) was added at room temperature and then powdered potassium carbonate (110 mmol) was added, followed by mixing with stirring for one hour. After removing the solvent using a vacuum evaporator, water (100 ml) and ethylacetate (100 ml) were added to the residue to separate an organic layer. The organic layer was dried with magnesium sulfate, and filtered. After removing the organic solvent from the filtrate using a vacuum evaporator, the residue was subjected to column chromatography, thus giving a desired 5-chloromethylfuran-2-carboxylic acid methylester with a yield of 50-70%.

(4-1) Preparation of 5-substituted oxymethylfuran-2-carboxylic Acid Ester, Amide (Compounds 58 to 191)

Potassium carbonate (1 mmol) was added to a mixture of 5-chloromethylfurancarboxilic acid ester (1 mmol), an alcoholic or amine compound (1-2 mmol) and acetonitrile (10 ml), and the reaction mixture was stirred for 5 hrs at room temperature. After checking completion of the reaction by thin layer chromatography, the solvent was removed from the reaction solution using a vacuum evaporator. Then, ethylacetate (10 ml) and water (10 ml) were added to the residue. The separated organic layer was dried and filtered, and subjected to column chromatography, thus giving a desired compound with a yield of 50-70%.

NMR data and mass spectra of the obtained compounds 1 and 31 to 57 are summarized below.

| Compound | NMR spectrum |
|---|---|
| No. | (200 MHz, CDCl₃, ppm) |
| 1 | 3.29 (s, 1H), 4.71 (s, 2H), 6.52 (d, 2H, J = 2.1 Hz), |
| | 7.23 (d, 2H, J = 2.1 Hz), 9.57 (s, 1H). |
| 31 | 9.66 (s, 1H), 8.03-8.08 (m, 2H), 7.34-7.62 (m, 3H), |
| | 7.23 (d, J = 3.5, 1H), 6.67 (d, J = 3.4, 1H), 5.38 (s, 2H) |
| 32 | 9.66 (s, 1H), 8.03-8.08 (m, 2H), 7.34-7.62 (m, 3H), |
| | 7.23 (d, J = 3.5, 1H), 6.67 (d, J = 3.4, 1H), 5.38 (s, 2H) |
| 33 | 9.64 (s, 1H), 7.20-7.33 (m, 1H), 7.21 (d, J = 3.6, |
| | 1H), 6.84-6.96 (m, 2H), 6.57 (d, J = 3.4, 1H), 5.19 (s, |
| | 2H), 3.77 (s, 2H) |
| 34 | 9.64 (s, 1H), 6.97-7.26 (m, 4H), 6.57 (d, J = 3.4, |
| | 1H), 5.19 (s, 2H), 3.89 (s, J = 1.6, 2H) |
| 35 | 9.64 (s, 1H), 7.12-7.32 (m, 5H), 6.55 (d, J = 3.6, |
| | 1H), 5.16 (s, 2H), 3.65 (s, 2H) |
| 36 | 9.64 (s, 1H), 7.19-7.26 (m, 2H), 6.92-6.96 (m, 2H), |
| | 6.57 (d, J = 3.4, 1H), 5.18 (s, 2H), 3.89 (s, 2H) |
| 37 | 9.64 (s, 1H), 7.23-7.34 (m, 1H), 7.19 (d, J = 3.4, |
| | 1H), 6.94-7.06 (m, 2H), 6.54 (d, J = 3.4, 1H), 5.16 (s, |
| | 2H), 3.67 (s, 2H) |
| 38 | 9.63 (s, 1H), 7.38-7.84 (m, 7H), 7.18 (d, J = 3.6, |
| | 1H), 6.54 (d, J = 3.6, 1H), 5.18 (s, 2H), 3.85 (s, 2H) |
| 39 | 9.66 (s, 1H), 7.17-7.28 (m, 1H), 7.20 (d, J = 3.6, |
| | 1H), 6.77-6.90 (m, 2H), 6.57 (d, J = 3.6, 1H), 5.17 (s, |
| | 2H), 3.69 (s, 2H) |
| 40 | 9.58 (s, 1H), 7.11-7.29 (m, 16H), 6.35 (d, J = 3.6, |
| | 1H), 5.27 (s, 2H) |
| 41 | 9.63 (s, 1H), 7.19-7.25 (m, 2H), 7.20 (d, J = 3.4, |
| | 1H), 6.89-6.91 (m, 2H), 6.54 (d, J = 3.4, 1H), 5.15 (s, |
| | 2H), 3.77 (s, 3H), 3.67 (s, 2H), |
| 42 | 9.63 (s, 1H), 7.23-7.81 (m, 1H), 7.19 (d, J = 3.6, |
| | 1H), 6.80-6.87 (m, 3H), 6.53 (d, J = 3.6, 1H), 5.15 (s, |
| | 2H), 3.78 (s, 3H), 3.64 (s, 2H) |
| 43 | 9.65 (s, 1H), 7.22 (d, J = 3.6, 1H), 6.33 (d, J = 3.6, |
| | 1H), 5.22 (s, 2H), 4.27 (t, J = 7.7, 1H), 2.05 (m, 2H), |
| | 1.18-1.31 (m, 22H), 0.87 (t, J = 6.3, 3H) |
| 44 | δ 9.64 (s, 1H), 7.20 (d, J = 3.6, 1H), 6.57 (d, J = 3.4, |
| | 1H), 5.12 (s, 2H), 2.35 (t, J = 7.2, 2H), 1.59-1.66 (m, |
| | 2H), 1.18-1.25 (m, 16H), 0.87 (t, J = 6.7, 3H) |
| 45 | 9.64 (s, 1H), 7.28-7.36 (m, 2H), 7.23 (d, J = 3.4, |
| | 1H), 6.97-7.05 (m, 2H), 6.70 (s, 1H), 6.63 (d, J = 3.4, |
| | 1H), 5.21 (s, 2H) |
| | Mass(m/s(intensity)): 263 (M), 248 (1), 219 (3), |
| | 190 (10), 137 (20), 109 (100), 80 (38), 53 (33) |
| 46 | 9.61 (s, 1H), 7.02-7.42 (m, 5H), 7.20 (d, J = 3.5, 1H), |
| | 7.19 (s, 1H), 6.60 (d, J = 3.5, 1H), 5.71 (s, 2H) |
| 47 | 9.63 (s, 1H), 7.20 (d, J = 3.5, 1H), 6.60 (d, J = 3.5, |
| | 1H), 5.10 (s, 2H), 4.59 (s, 1H), 3.80 (m, 1H), 1.18 (s, |
| | 1H), 1.14 (s, 1H) |
| 48 | 9.65 (s, 1H), 7.28-7.71 (m, 4H), 7.23 (d, J = 3.4, |
| | 1H), 6.89 (s, 1H), 6.65 (d, J = 3.4, 1H), 5.22 (s, 2H). |
| | Mass(m/s(intensity)): 313 (M, 1), 294 (1), 219 (3), |
| | 269 (1), 240 (2), 187 (22), 160 (36), 109 (100), 81 (29), |
| | 53 (22) |
| 49 | 9.65 (s, 1H), 7.51 (m, 1H), 7.23 (d, J = 3.6, 1H), |
| | 7.16-7.20 (m, 2H), 7.03-7.17 (m, 2H), 6.73 (s, 1H), |
| | 6.65 (d, J = 3.6, 1H), 5.22 (s, 2H) |
| | Mass(m/s(intensity)): 279 (M, 2), 235 (1), 206 (7), |
| | 153 (35), 125 (21), 109 (100), 89 (27), 80 (38), 63 (17), |
| | 53 (28) |
| 50 | 9.65 (s, 1H), 7.25-7.35 (m, 4H), 7.23 (d, J = 3.6, 1H), |
| | 6.71 (s, 1H), 6.64 (d, J = 3.6, 1H), 5.21 (s, 2H) |
| | Mass(m/s(intensity)): 324 (M, 4), 281 (2), 251 (7), |
| | 250 (7), 199 (22), 197 (21), 170 (13), 126 (2), |
| | 109 (100), 90 (30), 80 (30), 63 (20), 53 (27) |
| 51 | 9.65 (s, 1H), 7.87 (m, 1H), 7.21 (d, J = 3.2, 1H), |
| | 7.14 (m, 1H), 7.07 (s, 1H), 6.63 (d, J = 3.6, 1H), |
| | 6.46-6.50 (m, 1H), 5.21 (s, 2H), 3.82 (s, 3H), 3.78 (s, 3H) |
| | Mass(m/s(intensity)): 305 (M, 30), 179 (32), 152 (71), |
| | 124 (32), 109 (100), 93 (25), 81 (28), 53 (33) |
| 52 | 9.63 (s, 1H), 7.20 (d, J = 3.7, 1H), 6.57 (d, J = 3.6, |
| | 1H), 5.10 (s, 2H), 4.65 (s, 1H), 3.46 (s, 1H), |
| | 1.10-1.95 (m, 10H) |
| | Mass(m/s(intensity)): 251 (M), 224, 178 (1), |
| | 126 (100), 109 (52), 80 (19), 53 (19) |
| 53 | 9.63 (s, 1H), 7.20 (d, J = 3.7, 1H), 6.58 (d, J = 3.4, |
| | 1H), 5.11 (s, 2H), 4.76 (s, 1H), 3.18 (q, J = 6.5, 2H), |
| | 1.25-1.53 (m, 4H), 0.92 (t, J = 6.5, 3H) |
| | Mass(m/s(intensity)): 225 (M+), 152, 126 (100), |
| | 109 (34), 80 (16), 53 (16) |
| 54 | 9.65 (s, 1H), 7.15-.38 (m, 3H), 7.23 (d, J = 3.6, 1H), |
| | 6.73 (s, 1H), 6.65 (d, J = 3.7, 1H), 5.22 (s, 2H) |
| | Mass(m/s(intensity)): 313 (M), 240 (1), 187 (47), |
| | 159 (13), 124 (47), 109 (100), 80 (29), 53 (33), 41 (36) |
| 55 | 9.67 (s, 1H), 8.12-8.16 (m, 1H), 6.98-7.38 (m, 3H), |
| | 7.23 (d, J = 3.2, 1H), 6.66 (d, J = 3.2, 1H), 5.25 (s, 2H) |
| | Mass(m/s(intensity)): 279 (M, 5), 235 (5), 153 (28), |
| | 125 (22), 109 (100), 80 (33), 53 (31) |
| 56 | 9.64 (s, 1H), 7.23 (d, J = 3.6, 1H), 6.63 (d, J = 3.7, |
| | 1H), 5.12 (s, 2H), 4.74 (s, 1H), 3.24 (q, J = 6.9, 2H), |
| | 1.14 (t, J = 7.4, 3H) |
| | Mass(m/s(intensity)): 197 (M, 63), 169 (43), |
| | 109 (100), 90 (90), 81 (26), 69 (38), 63 (59), 53 (38), |
| | 41 (92) |
| 57 | 9.64 (s, 1H), 7.41-7.88 (m, 7H), 7.21 (d, J = 3.4, 1H), |
| | 7.12 (s, 1H), 6.64 (d, J = 3.5, 1H), 5.25 (s, 2H) |
| | Mass(m/s(intensity)): 295 (M, 8), 251 (6), 222 (14), |
| | 194, 169 (100), 140 (51), 126 (20), 115 (45), 109 (69), |
| | 80 (38), 53 (33), 41 (39) |

### Experimental Example 1

### Evaluation of Effects of the Compound (Compound 1) of the Present Invention on Proliferation, Differentiation and Activity of Osteoblasts

The compound (Compound 1) of the present invention was evaluated for effects on osteoblast proliferation, differentiation and activity in the following tests.

In the following tests, three kinds of cell lines were used. The human osteosarcoma cell lines, MG-63 (ATCC No. CRL-1427) and HOS (ATCC No. CRL-1543), and mouse muscle C2C12 muscle cells (ATCC No. CRL-1772) were purchased from ATCC (American Type Culture Collection, Rockville, USA), and cultured in Dulbecco's modified Eagle's medium (DMEM) supplemented with 10% fetal bovine serum (FBS).

### 1-1. Evaluation of Effects of the Compound (Compound 1) of The Present Invention on Proliferation of Osteoblasts

In order to indirectly evaluate cytotoxicity of the Compound 1 and investigate effect of the Compound 1 naturally extracted on obsteoblast proliferation, proliferation of MG-63 osteoblasts was investigated by measuring the amount of ³H-thymidine incorporated into cellular DNA upon DNA replication. The cells were aliquotted onto 24-well plates at a density of 2×10⁴ cells per well. The next day, after exchanging culture medium to medium containing 1% FBS, the cells were treated with the compound 1 serially diluted at various concentrations and further cultured for 48 hrs. Four hours before finishing the culture of 48 hrs, ³H-thymidine was added to the medium in an amount of 3 μCi per well. The cells were washed with phosphate buffer, and treated with 5% trichloroacetic acid (TCA) pre-cooled on ice. Each of the separated TCA-insoluble fractions was dissolved in 0.1 M NaOH. With a portion of each fraction, ³H-thymidine uptake by the cells was measured using a liquid scintillation counter. The results are given in Table 8, below, and FIG. 1.

TABLE 8

| | Proliferation rate |
|---|---|
| Conc. of Compound 1, M | of MG-63 cells, % |
| 0 | 100.00 ± 8.30 |
| 10⁻⁸ | 101.38 ± 4.65 |
| 10⁻⁷ | 103.91 ± 5.81 |
| 10⁻⁶ | 105.56 ± 5.49 |
| 10⁻⁵ | 102.63 ± 8.53 |
| 10⁻⁴ | 105.50 ± 5.90 |

To achieve recovery from osteoporosis, osteoblasts that are responsible for bone formation in bone tissue should be primarily proliferated. In this test, as described above, when osteoblasts were treated with the Compound 1, the number of osteoblasts was investigated. As a result, the cells treated with the Compound 1 of the present invention showed a proliferation rate of over 105%, compared to a control not treated with the Compound 1 (Table 8 and FIG. 1). These results indicate that the compound of the present invention has a stimulatory effect on proliferation of osteoblasts.

### 1-2. Assay for Activity of Alkaline Phosphatase (Osteoblast Marker Enzyme)

To examine whether the Compound 1 affects positively or negatively osteoblast activity, alkaline phosphatase (ALP) activity was measured. HOS cells were aliquotted onto a 96-well microplate at a density of 5×10³ cells per well, and cultured. When forming a monolayer, the cells were treated with the naturally extracted compound (Compound 1) at prescribed concentrations. After incubation in 10% FBS-containing DMEM for 48 hrs, the cells were treated with 0.1% Triton X-100/saline for 30 min at 37° C. A portion of each cell lysate was reacted with 0.1 N glycine-NaCh buffer (pH 10.4) for 30 min at 37° C. in the presence of 100 mM nitrophenyl phosphate (PNPP) as a substrate of alkaline phosphate. The amount of PNP (ρ-nitrophenol) released from the substrate PNPP was determined by measuring absorbance at 405 nm. Protein amount was determined by a modified Lowry method, and enzyme activity was designated as nmol substrate cleaved/h/mg protein. The results are given in Table 9 and FIG. 2.

TABLE 9

| Conc. of Compound 1, M | ALP activity in HOS cells, % |
|---|---|
| 0 | 100.00(6.82 |
| 10-8 | 117.05(3.13 |
| 10-7 | 121.16(4.93 |
| 10-6 | 115.76(2.67 |

Alkaline phosphatase, used as an osteoblast marker, was reported to participate in several steps of bone formation (Siffert, 1958; Farley and Baylink, 1986). In this test, as described above, alkaline phosphate activity as an indicator of osteoblast activity was determined in order to investigate effect of the Compound 1 on osteoblast activity. As a result, when the cells were treated with the Compound 1, alkaline phosphate activity was elevated to about 121%, compared to a control not treated with the Compound 1 (Table 9 and FIG. 2). These results, in which the Compound 1 has excellent effects on the activity of alkaline phosphatase as an osteoblast marker enzyme, indicate that the compound of the present invention has a stimulatory effect on osteoblast activity.

### 1-3. Evaluation of Expression of Transcriptional Factor Runx2 (a Key Regulator of Osteoblast Differentiation)

Primarily, transient transfection was carried out using a p6xOSE2-Luc vector, which was prepared by inserting into a pGL3 promoter vector six tandem copies of the OSE2 motif, the binding site of the transcriptional factor Runx2 that is a critical regulator for osteoblast differentiation. Runx2 expression levels were determined by luciferase assay. The test procedure will be described in more detail, as follows.

C2C12 cells were plated onto 6-well plates at a density of 1 (105 cells per well, and cultured for 24 hrs in a 5% CO2 incubator.

Then, the cells transiently transfected with the p6xOSE2-Luc vector using a transfection reagent, LipofectAmine, as follows. The plasmid DNA was reacted with the transfection reagent for 15 min to form p6xOSE2-Luc DNA:lipofectamine complexes. During this reaction, culture medium was removed from each well of the plates, the cells were washed with FBS-free DMEM, and 800 μl of FBS-free DMEM was added to each well. 207 μl of the formed p6xOSE2-Luc DNA:lipofectamine complexes was added to each well, followed by incubation for 3 hrs in a 5% CO₂ incubator. Then, 1 ml of 30% FBS-containing DMEM was added to each well, and the cells were further cultured for 24 hrs in a 5% CO₂ incubator.

After removing the medium from each well and washing the cells with FBS-free DMEM, 2 ml of FBS-free DMEM containing 10 ng/ml of basic fibroblast growth factor (bFGF) was added to each well. The cells not treated with bFGF were used as a control. After 24 hrs of incubation in a 5% CO₂ incubator, the cells were washed with PBS and applied to luciferase assay. The results are given in Table 10 and FIG. 3.

TABLE 10

| Conc. of Compound 1, μM | Expression level of Runx2, % |
|---|---|
| 0 | 100.0 |
| 1 | 209.2 |
| 10 | 218.9 |

As a result, when treated with 1 and 10 μM of the Compound 1, the cells showed elevated Runx2 activity to over 200%, compared to the control (Table 10 and FIG. 3). In order to stimulate bone formation, osteoblasts should be primarily differentiated. Expression of the transcription factor Runx2 is essential for stimulation of obsteoblast differentiation, and resulted in an increase of expression of some genes (osteocalcin, osteopontin, type I collagen and bone sialoprotein) of which translational products are required for osteoblast differentiation. These genes have promoters containing a Runx2-binding site, that is, OSE (osteoblast specific factor binding element) with a consensus sequence of PuACCPuCA (Lee et al., 2000; Park et al., 2001). In this regard, the elevated expression level of Runx2 may lead to increased levels of the proteins required for bone formation, and eventually, stimulation of bone formation.

### 1-4. Evaluation of Expression of Osteoprotegerin (OPG) Inhibiting Osteoclast Production

When MG63 cells grew confluently on 96-well plates, 2 ml of FBS-free DMEM containing various concentrations of the Compound 1 was added to each well. After incubation for 16 hrs, the culture supernatants were collected. The amount of OPG released into the medium was determined using an OPG ELISA kit. The results are given in Table 11, below, and FIG. 4.

TABLE 11

| Conc. of Compound 1, M | OPG expression level in MG63 cells, % |
|---|---|
| 0 | 100.00 ± 0.00 |
| 10⁻¹⁰ | 185.36 ± 30.56 |
| 10⁻⁸ | 204.30 ± 14.96 |
| 10⁻⁶ | 866.90 ± 10.59 |

Receptor activator of NF-κB receptor (RANKL) is essentially required for production and activation of osteoclasts. RANKL is expressed in osteoblasts and mesenchymal cells, and binds to receptor activator of NF-κB (RANK) in osteoclast progenitors and osteoclasts (Hofbauer et al., 2000). OPG, as a decoy receptor of RANKL, interferes with RANK-RANKL binding, and thus inhibits differentiation of osteoclast progenitors to osteoclasts and the activity of osteoclasts (Kong et al., 1999; Yasuda et al., 1999; Suda et al., 1999; Aubin et al., 2000). This test was performed to determine whether the Compound 1 increases secretion of OPG by osteoblasts.

As a result, when the MG63 osteoblasts were treated with 10⁻¹⁰, 10⁻⁸ and 10⁻⁶ M of the Compound 1 of the present invention, expression of OPG inhibiting osteoclast formation was increased to about 185%, 204% and 866%, respectively (Table 11 and FIG. 4). These results demonstrated that the Compound 1 of the present invention inhibits osteoclast formation by stimulating expression of OPG inhibiting osteoclast formation in osteoblasts.

### Experimental Example 2

### Inhibitory Effect of Compound 1 on Proliferation of Osteoclasts

In order to investigate the effects of the Compound 1 on differentiation and activity of osteoclasts, when differentiation of osteoclast progenitors was induced, the activity of Tartrate-resistant acid phosphatase (TRAP) as an osteoclastic marker enzyme was investigated, and, when differentiated osteoclasts were cultured on calcium phosphate-coated plates (OAAS, OCT Inc.), resorption activity (formation of resorption pits) were measured.

2-1. Isolation of Osteoclast Progenitors and Induction of their Differentiation to Mature Osteoclasts

First, bone marrow cells were isolated, as follow. After sacrificing 7-9 week female mice by cervical dislocation, femur and tibia were excised aseptically while removing attached soft tissues. After cutting both ends of long bones, 1 ml of an enzyme solution, containing 0.1% collagenase (Gibco), 0.05% trypsin and 0.5 mM EDTA (Gibco), was injected to the bone marrow cavity at one end using a syringe with a 26-guage needle, and bone marrow was then collected. After shaking the recovered bone marrow for 30 min, bone marrow cells were collected, and cultured in α-minimum essential medium (α-MEM) supplemented with 10% FBS for 24 hrs. Then, non-adherent cells were collected, which are osteoclast pregenitors, aliquotted onto culture plates at a density of 2×10⁵ cells per well, and cultured for 8 days in α-MEM supplemented with 20 ng/ml of macrophage-colony stimulating factor (M-CSF, Peprotech, USA) and 30 ng/ml RANKL (Peprotech, USA). During the 8 days of culturing, the cells were treated with the Compound 1. Thereafter, osteoclast formation was investigated by fixing the cultured cells and then carrying out TRAP staining, as described in the following Experimental Example 2.2. In addition, resorption activity of osteoclasts was investigated by measuring the absorption area after removing osteoclasts in cultures in calcium phosphate-coated plates, as described in Experimental Example 2.3, below.

### 2-2. Evaluation of Formation of Trap-Positive Multinucleated Cells

After cell culture, the adherent cells were washed with PBS, and TRAP staining was performed, as follows. After being fixed with citrate-acetate-formaldehyde for 5 min, the cells were incubated for 1 hr in acetate buffer (prewarmed at 37° C., pH 5.0) containing naphthol AS-BI phosphate, fast Garnet GBC and 7 mM tartrate buffer (pH 5.0). TRAP-positive multinucleated cells having 3 or more nuclei were considered as osteoclasts. The results are given in Table 12, below, and FIG. 5.

TABLE 12

| Conc. of Compound 1, M | No. of TRAP(+) multinucleated cells |
|---|---|
| 0 | 132.80 ± 10.50 |
| 10⁻⁸ | 105.20 ± 8.58 |
| 10⁻⁷ | 120.00 ± 6.42 |
| 10⁻⁶ | 103.60 ± 6.95 |
| 10⁻⁵ | 108.20 ± 4.57 |
| 10⁻⁴ | 77.20 ± 6.73 |

Osteoclasts are derived from hematopoietic cells belonging to the monocyte/macrophage lineage originating in bone marrow. These mononucleated progenitor cells are carried by the bloodstream, and proliferate in the inner side of bone and fuse together to become multinucleated (Scheven et al., 1986). Osteoclasts secrete tartrate-resistant acid phosphate (TRAP) that is used as a biochemical marker of osteoclasts while allowing discrimination of osteoclasts from other cells (Minkin, 1982). In this test, bone marrow known to contain progenitor cells of osteoclasts was used to induce differentiation of osteoclasts, and treated with the Compound 1 of the present invention for 8 days. The formed TRAP-positive multinucleated cells containing three or more nuclei were counted as osteoclasts, and the counted cell number was compared to that of a control not treated with the Compound 1.

As a result, compared to the control, the number of TRAP-positive multinucleated cells in cultures treated with the Compound 1 was remarkably reduced from 132 to 77 (FIG. 5). These results indicate that the Compound 1 of the present invention, prepared in Example 1, has an inhibitory effect on osteoclast formation.

### 2-3. Evaluation of Resorption Activity of Osteoclasts

The differentiated osteoclasts were cultured on calcium phosphate-coated plates (OAASTM, OCT, Korea) in order to evaluate resorption activity of the cells. After removing culture medium, to remove the adherent cells, the plates were washed with distilled water three times, and incubated in 5% sodium hypochlorite for 5 min. After washing again with distilled water and drying the plates, the area of formed resorption pits was calculated by means of an Image Pro Plus software. The results are given in Table 13, below, and FIG. 6.

TABLE 13

| Conc. of Compound 1, M | Resorption activity of osteoclasts, % |
|---|---|
| 0 | 100.00 ± 9.67 |
| 10⁻⁸ | 66.03 ± 7.49 |
| 10⁻⁷ | 61.46 ± 10.35 |
| 10⁻⁶ | 41.75 ± 14.07 |
| 10⁻⁵ | 46.92 ± 3.60 |
| 10⁻⁴ | 52.34 ± 8.28 |

In this test, in order to evaluate the activity of osteoclasts responsible for bone absorption in bone tissue, differentiated osteoclasts were cultured in plates coated with calcium phosphate, where the formed matrix is similar to the inorganic portion of bone tissue (Choi et al., 2001), while being treated with the Compound 1. The area of the formed absorption pits was compared to a control not treated with the Compound 1.

As a result, compared to the control, the area of resorption pits in cultures treated the Compound 1 was reduced to about 50% (Table 12 and FIG. 6). These results indicate that the Compound 1 has an effect of inhibiting the activity of osteoclasts.

### 2-4. Evaluation of Inhibition Rates of Furan Derivatives of the Present Invention Versus Osteoclast Formation

Osteoclast pregenitor cells were isolated, and their differentiation to mature osteoclasts was induced, according to the same method as in the Experimental Example 2-1, while treated with each of the furan derivatives of the present invention. According to the same method as in the Experimental Example 2-2, TRAP staining performed, and TRAP-positive multinucleated cells were counted. An inhibition rate of each furan derivative against osteoclast formation was expressed as percentage, wherein the number of stained cells in a control not treated the furan derivative was arbitrarily set as 100% (Tables 14 to 16).

TABLE 14

| | | | Inhibition rate against | | |
|---|---|---|---|---|---|
| Compound | | | osteoclast formation, % | | |
| No. | X | Y | 0.1 μM | 1 μM | 10 μM |
| 1 | H | HO— | 0 | 0 | 30 |
| 32 | H | C₆H₅CH₂COO— | 0 | 23.3 | 50 |
| 33 | H | 2,6-F₂C₆H₃CH₂COO— | 0 | 3.3 | 50 |
| 34 | H | 2-Cl-6-FC₆H₃CH₂COO— | 0 | 16.7 | 36.7 |
| 35 | H | 3-Cl—C₆H₄CH₂COO— | 0 | 23.33 | 43.33 |
| 36 | H | 3-SC₄H₃CH₂COO— | 0 | 6.67 | 30 |
| 37 | H | 3-F—C₆H₄CH₂COO— | 0 | 10 | 6.67 |
| 38 | H | 2-NpCH₂COO— | 0 | 23.33 | 20 |
| 39 | H | 2,4-F₂C₆H₃CH₂COO— | 0 | 10 | 56.67 |
| 40 | H | (C₆H₅)₃CCOO— | 0 | 16.67 | 43.33 |
| 41 | H | 2-CH₃O-6- | 0 | 6.67 | 56.67 |
| | | FC₆H₃CH₂COO— | | | |
| 42 | H | 3-CH₃O-6-FC₆H₃CH₂CO— | 0 | 23.33 | 53.33 |
| 43 | H | 2-BrC₁₄H₂₈COO— | 0 | 17.6 | 90.26 |
| 44 | H | C₁₄H₂₉COO— | 0 | 16.67 | 53.33 |
| 49 | H | 3-ClC₆H₄NHCOO— | 0 | 10 | 0 |
| 50 | H | 4-BrC₆H₄NHCOO— | 0 | 10 | 0 |
| 53 | H | CH₃CH₂CH₂CH₂NHCOO— | 0 | 10 | 36.67 |
| 54 | H | 3,4-Cl₂C₆H₃NHCOO— | 0 | 0 | 10 |
| 55 | H | 2-ClC₆H₄NHCOO— | 0 | 20 | 20 |
| 59 | CH₃O— | 2-CH₃O-4- | 0 | 0 | 15.95 |
| | | CH₂═CHCH₂C₆H₃O— | | | |
| 60 | CH₃O— | 2,4-Cl₂C₆H₄O— | 0 | 0 | 32.75 |
| 61 | CH₃O— | 2-ClC₆H₄O— | 0 | 0 | 11.66 |
| 66 | CH₃O— | 2-NO₂-4-ClC₆H₃O— | 0 | 0 | 13.58 |
| 67 | CH₃O— | 2-NO₂C₆H₄O— | 0 | 0 | 15.92 |
| 68 | CH₃O— | 2-(CH₃)₂CH-5- | 0 | 0 | 5.62 |
| | | CH₃C₆H₃O— | | | |
| 70 | CH₃O— | 3,4-(CH₂)₃C₆H₃O— | 0 | 0 | 3.34 |
| 75 | CH₃O— | 3-CH₃-4-ClC₆H₃O— | 0 | 0 | 0.78 |
| 79 | CH₃O— | 4-IC₆H₄O— | 0 | 0 | 34.65 |
| 80 | CH₃O— | 4-ClC₆H₄O— | 0 | 0 | 15.19 |
| 85 | CH₃O— | (CH₃)₂N— | 0 | 0 | 3.75 |
| 89 | CH₃O— | C₆H₅NH— | 0 | 0 | 12.6 |
| 91 | CH₃O— | (CH₃)₃CNH— | 0 | 0 | 9.89 |
| 94 | (CH₃)₂CHO— | 2-CH₃OC₆H₄O— | 0 | 0 | 5.9 |
| 95 | (CH₃)₂CHO— | 2,5-Cl₂C₆H₃O— | 0 | 0 | 0.97 |
| 97 | 4-ClC₆H₄O— | 2-CH₃OC₆H₄O— | 0 | 0 | 12.75 |

TABLE 15

| | | | Inhibition rate against | | |
|---|---|---|---|---|---|
| Compound | | | osteoclast formation, % | | |
| No. | X | Y | 0.1 μM | 1 μM | 10 μM |
| 98 | C₆H₅O— | 2-ClC₆H₄O— | 0 | 0 | 9.89 |
| 100 | HO— | 4-CH₃CH₂CH₂C₆H₄O— | 0 | 0 | 10.46 |
| 102 | HO— | 4-FC₆H₄O— | 0 | 0 | 5.05 |
| 103 | HO— | 4-BrC₆H₄O— | 0 | 0 | 2.77 |
| 107 | HO— | 2-ClC₆H₄O— | 0 | 0 | 32 |
| 108 | HO— | 2-BrC₆H₄O— | 0 | 0 | 14.72 |
| 109 | HO— | 2,5-Cl₂C₆H₃O— | 0 | 0 | 3.25 |
| 112 | HO— | 3-ClC₆H₄O— | 0 | 0 | 7.79 |
| 114 | HO— | 4-(CH₃)₂CHC₆H₄O— | 0 | 0 | 4.2 |
| 117 | HO— | 1-NpO— | 0 | 0 | 5.86 |
| 119 | HO— | 4-Cl-3-CH₃C₆H₃O— | 0 | 0 | 37.02 |
| 120 | HO— | C₆H₅CH₂S— | 0 | 0 | 27.99 |
| 121 | CH₃CH₂CH(CH₃)NH— | 4-IC₆H₄O— | 0 | 0 | 7.12 |
| 123 | CH₃CH₂)₂N— | 4-Cl-3-CH₃C₆H₃O— | 0 | 6.9 | 100 |
| 124 | CH₃CH(CH₂CH₂)₂N— | 2,5-Cl₂C₆H₄O— | 0 | 0 | 7.62 |
| 127 | (CH₃)₃CNH— | CF₃CH₂O— | 0 | 0 | 13.82 |
| 128 | (CH₂)₆N— | 2-BrC₆H₄O— | 6.54 | 0 | 97.65 |
| 129 | (CH₃)₃CNH— | 2-BrC₆H₄O— | 0 | 0 | 2.2 |
| 130 | (CH₃)₂CHNH— | 2,5-Cl₂C₆H₃NH— | 0 | 0 | 7.84 |
| 132 | O(CH₂CH₂)₂N— | 3-CH₃-4-ClC₆H₃O— | 0 | 0 | 62.30 |
| 133 | (CH₂)₆N— | 2,5-Cl₂C₆H₃O— | 0 | 0 | 0.5 |
| 134 | (CH₂)₅CHNH— | 4-(CH₃)₃CC₆H₄O— | 0 | 0 | 21.42 |
| 136 | C₆H₅NH— | 4-(CH₃)₃CC₆H₄O— | 0 | 0 | 2.49 |
| 137 | C₆H₅NH— | 2-NpO—* | 0 | 0 | 38.3 |
| 140 | 3-BrC₆H₄NH— | 4-FC₆H₄O— | 0 | 0 | 11.93 |
| 141 | 4-FC₆H₄NH— | 3-ClC₆H₄O— | 0 | 0 | 13.01 |
| 142 | 3-Cl-4-CH₃OC₆H₃NH— | 2-ClC₆H₄O— | 0 | 0 | 32.17 |
| 143 | 3,4-F₂C₆H₃NH— | 4-IC₆H₄O— | 0 | 0 | 10.63 |
| 144 | 2-CH₃CH₂OC₆H₄NH— | 2-NO₂-4-ClC₆H₃O— | 0 | 21.95 | 85.25 |
| 145 | 2,4-(CH₃O)₂C₆H₃NH— | 2,5-Cl₂C₆H₃O— | 0 | 5.98 | 83.3 |
| 146 | 4-BrC₆H₄NH— | 4-IC₆H₄O— | 0 | 0 | 10.03 |
| 147 | 4-FC₆H₄NH— | 2-NO₂-4-CH₃C₆H₃O— | 6.11 | 19.4 | 99.72 |
| 148 | 4-NH₂COC₆H₄NH— | 2,5-Cl₂C₆H₃O— | 0 | 3.05 | 47.55 |
| 149 | 2-NO₂-4-CH₃OC₆H₃NH— | 4-F—C₆H₄O— | 0 | 10.32 | 78.34 |
| 150 | 4-CH₃OC₆H₄NH— | 3-CH₃-4-ClC₆H₃O— | 0 | 0 | 19.12 |

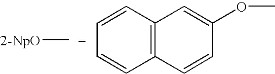

TABLE 16

| | | | Inhibition rate against | | |
|---|---|---|---|---|---|
| Compound | | | osteoclast formation, % | | |
| No. | X | Y | 0.1 μM | 1 μM | 10 μM |
| 151 | 2,5-F₂C₆H₃NH— | 3-ClC₆H₄O— | 0 | 0 | 19.8 |
| 152 | 2-CH₃-5- | 4-IC₆H₄O— | 5.37 | 0.08 | 37.56 |
| | CH₃O₂CC₆H₃NH— | | | | |
| 155 | 2-F-5-CH₃C₆H₃NH— | 2-NO₂-4-CH₃C₆H₃O— | 0 | 0 | 27.92 |
| 156 | 2,3-Cl₂C₆H₃NH— | 2-NO₂C₆H₄O— | 13.01 | 18.26 | 98.48 |
| 157 | 2-CH₃OC₆H₄NH— | 2,5-(CH₃)₂C₆H₃O— | 4.87 | 10.71 | 97.46 |
| 158 | 2-F-5-CH₃C₆H₃NH— | 2-NO₂C₆H₄O— | 0 | 0 | 23.57 |
| 160 | 4-CH₃COC₆H₄NH— | 1-NpO— | 0 | 0 | 38.96 |
| 161 | 2,5-F₂C₆H₃NH— | 2-ClC₆H₄O— | 0 | 0 | 11.33 |
| 163 | 3-CH₃CH₂C₆H₄NH— | 4-F—C₆H₄O— | 0 | 0 | 51.2 |
| 164 | 3,4- | 4-CH₃CH₂CH₂C₆H₄O— | 0 | 0 | 30.52 |
| | (CH₃O)₂C₆H₃NH— | | | | |
| 165 | (CH₃)₃CNH— | 4-CH₃CH₂OC₆H₄O— | 12.35 | 28.76 | 88.75 |
| 166 | 2-CH₃OC₆H₄NH— | 2-CH₃OC₆H₄O— | 0 | 0 | 1.08 |
| 167 | 2-CH₃O₂CC₆H₄NH— | 2,4-Cl₂C₆H₃O— | 0 | 22.42 | 96.47 |
| 169 | 3,5-(CH₃)₂C₆H₃NH— | 4-CH₃CH₂—C₆H₄O— | 0 | 0 | 21.04 |
| 170 | 2,5-Cl₂C₆H₃NH— | 4-CH₃CH₂CH₂C₆H₄O— | 0 | 0 | 16.31 |
| 171 | 2-CH₃O-5- | 3-ClC₆H₄O— | 0 | 0 | 0.42 |
| | CH₃C₆H₃NH— | | | | |
| 172 | 2,3-(CH₃)₂C₆H₃NH— | 3-CH₃-4-ClC₆H₃O— | 0 | 0 | 11.08 |
| 173 | 4-ClC₆H₄NH— | 3-CH₃-4-ClC₆H₄O— | 0 | 0 | 8.03 |
| 174 | 2-ClC₆H₄NH— | 2-CH₃OC₆H₄O— | 0 | 0 | 14.11 |
| 175 | 3,4-(CH₃)₂C₆H₄NH— | 2-CH₃OC₆H₄O— | 0 | 0 | 33.33 |
| 176 | 2,4-F₂C₆H₃NH— | 2-CH₃O-4- | 0 | 0 | 15.56 |
| | | CH₃CH₂CH₂C₆H₃O— | | | |
| 177 | 2-FC₆H₄NH— | 3,5-(CH₃)₂C₆H₃O— | 0 | 0 | 11.03 |
| 178 | 2-FC₆H₄NH— | 4-ClC₆H₄O— | 0 | 0 | 14.09 |
| 179 | 2,6-(CH₃)₂C₆H₃NH— | 4-ClC₆H₄O— | 0 | 0 | 34.81 |
| 180 | 2-CH₃O₂CC₆H₄NH— | 3,4-(CH₃)₂C₆H₃O— | 0 | 0 | 8.15 |
| 181 | 2-Cl-5-CF₃C₆H₃NH— | 4-ClC₆H₄O— | 0 | 0 | 56.96 |
| 182 | 2-CH₃O-4- | 4-ClC₆H₄O— | 0 | 0 | 4.34 |
| | NO₂C₆H₃NH— | | | | |
| 183 | 2-NO₂C₆H₄NH— | 2-ClC₆H₄O— | 0 | 0 | 57.39 |
| 185 | 2,4-F₂C₆H₃NH— | 2,3,5,6-F₄C₆HO— | 0 | 0 | 13.64 |
| 186 | 3,4-F₂C₆H₃NH— | 3,4-(CH₃)₂C₆H₃O— | 0 | 0 | 21.09 |
| 187 | 3-CH₃CONHC₆H₄NH— | C₆F₅O— | 21.4 | 37.05 | 36.74 |
| 188 | 2,4- | 2-BrC₆H₄O— | 0 | 0 | 7.6 |
| | (CH₃O)₂C₆H₄NH— | | | | |
| 189 | 2-CH₃OC₆H₄NH— | 4-(CH₃)₃CC₆H₄O— | 0 | 0 | 48.99 |
| 190 | 4-IC₆H₄NH— | 2-BrC₆H₄O— | 0 | 0 | 5.12 |
| 191 | 3-NO₂C₆H₄NH— | 4-IC₆H₄O— | 0 | 0 | 8.06 |

As shown in the above Tables, the furan derivatives of the present invention have an inhibitory effect versus osteoclast formation. In detail, when used in an amount 0.1 μM, the compounds 128, 147, 152, 156, 157, 165 and 187 had an inhibition rate of 4-21% versus osteoclast formation. When used in an amount 10 μM, most of the furan derivatives showed inhibitory effects versus osteoclast formation.

### Experimental Example 3

### Clinical Tests with an Animal Model with Osteoporosis

In order to evaluate effects of the compound of the present invention on treatment and prevention of osteoporosis, female white mice with osteoporosis induced by ovariectomy were used as a disease model. After ovariectomy, mice were administered with the compound of the present invention. Tibias were dissected from the mice, and observed histologically.

### 3-1. Ovariectomy

After anesthetizing female white mice with 100 mg/kg of Ketamine (Ketara) and 0.15 ml/kg of 2% Xylazine (Rompun), the lumbar dorsum is shaved bilaterally and the exposed skin was prepared for aseptic surgery by a 10% povidone-iodine scrub followed by a 70% alcohol wipe.

A 1-cm incision was made in the central region of the abdomen, and the ovary was identified along with the uterus with caution not to damage the main organs such as the liver and diaphragm. After ligating the uterus with a thread for suturing, the exposed ovary was severed toward both sides and removed. Thereafter, each organ was relocated to its original position, and the incision was closed with a thread for suturing in an interrupted pattern. After ovariectomy, the mice were injected with 50 mg/kg of cefazolin antibiotic for prevention of infection with pathogens.

### 3-2. Administration of the Compound 1

The daily dosage of the Compound 1 per female white mouse was determined based on 250 g body weight. In detail, a control group was fed with solid feedstuff and 30 ml water per mouse. A test group was administered with a mixture of 100 mg of the Compound 1 and 30 ml of water per mouse, by using a water bottle. This mixture was prepared every morning, and the remaining mixture was not discarded and mixed with a freshly prepared one next morning, wherein the fresh mixture was added to the water bottle regardless of the remaining amount according to the mouse number in a cage. Water was supplemented everyday, based on the number of mice in each group. With caution not to exchange the water bottles between the groups, everyday, the number of mice was recorded, and the mixture was prepared based on the number of mice.

In order to evaluate effect of the Compound 1 on prevention of osteoporosis, immediately after ovariectomy, the mice were administered with the Compound 1 contained in water for four weeks, as described above. Then, 16 mice from the test group and four mice from the control group were randomly selected. After checking change in body weight of the mice, tibia tissue specimens were fixed, dehydrated, cleared and stained for histological studies, according to a method known in the art.

In order to evaluate therapeutic effect of the Compound 1 on osteoporosis, after ovariectomy, the mice were fed with solid feedstuff for four weeks. After checking progress of osteoporosis in the mice, a test group was administered with the Compound 1 for four weeks, according to the same method as described above, while a control group was fed with only solid feedstuff. Eight weeks after ovariectomy, tibia tissue specimens were prepared according to a method known the art.

### 3-3. Observation of the Bone Tissue Specimens Under Light Microscope

The tibia tissue specimens prepared in the Experimental Example 3-2 were fixed in a Bouin's solution for about 24 hrs. Then, the tibia tissue specimens were decalcificated with 5% nitric acid for about 60 hrs in order to remove inorganic substances including calcium from the tibias, thus softening the tibias to the degree suitable for fracture. Thereafter, the tibia tissue specimens were washed with running water for 12 hrs, sequentially dehydrated in 70%, 80%, 90%, 95% and 100% ethanol for 2 hrs three times per each case, and cleared with xylene for 2 hrs three times. Finally, the tibia tissue specimens were embedded in paraffin by being immersed in liquid paraffin for 2 hrs three times to allow penetration of paraffin into the tibias.

Paraffin blocks were slice-cut using a rotatory microtome. The obtained 4-μm sections of the tibias were mounted on slide glasses coated with Poly-L-lysin, and dried by using a slide warmer (40±3° C.). The completely dried slides were deparaffinized in xylene, dehydrated in alcohol, and stained with either hematoxylin-Eosin or Gomori's trichrome. The stained tissues were rinsed in alcohol and cleared in xylene and alcohol. Then, the slides were covered with a covering agent, and dried for 24 hrs in an incubator at 60° C., followed by light microscopic examination and capturing of the microscopic images.

### 3-4. Preventive Effect 1 of the Compound 1 on Osteoporosis

20 female white mice weighing about 200 g were divided into a control group (4 mice) and a test group (16 mice) After performing ovariectomy according to the same method as described above, the mice of the test and control groups were administered with the Compound 1 for four weeks or not, under the same conditions as described above. After checking change in body weight of the mice, tibia tissue specimens were fixed, dehydrated, cleared and stained for histological studies, according to a method known in the art.

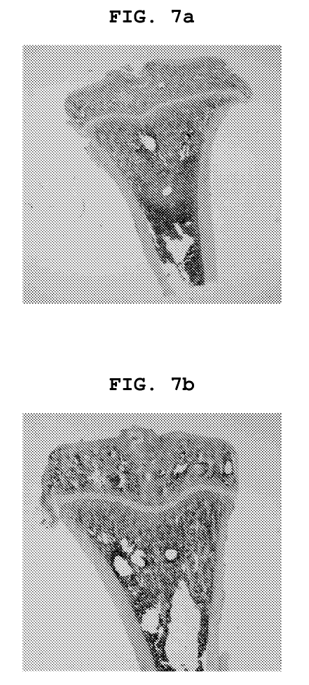

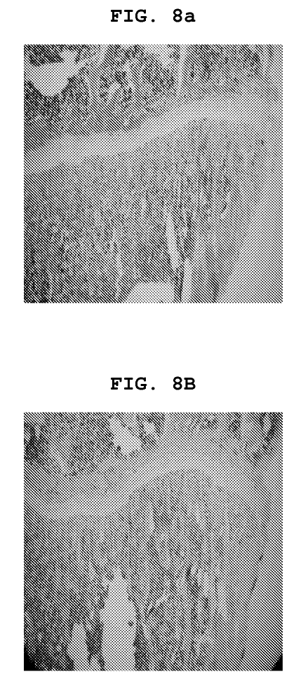

As a result of microscopic examination with 12.5× magnification, the trabecular bone (the porous inner core of the vertebra) and the cortical bone that is the outer sheath surrounding the core were lost in the control group not administered with the Compound 1 (FIG. 7a), whereas such trabecular bone loss was not observed in the test group administered with the Compound 1 (FIG. 7b). Cross-sectional view of tibias under a microscope with 40× magnification revealed a marked increase in the cortical and trabecualr bone in the test group, compared to the control group, while the bone marrow cavity in the tibia from the test group was filled with trabecular bone (FIGS. 8a and 8b).

### 3-5. Preventive Effect 2 of the Compound 1 on Osteoporosis

20 female white mice weighing about 200 g were divided into a control group (4 mice) and a test group (16 mice), and administered with the Compound 1 for four weeks or not. After being dissolved in 1 ml of physiological saline, 10 mg of the Compound 1 was subcutaneously injected into the mice of the test group. The mice of the control group were subcutaneously injected with 1 ml of physiological saline. After checking change in body weight of the mice, tibia tissue specimens were fixed, dehydrated, cleared and stained for histological studies, according to a method known in the art.

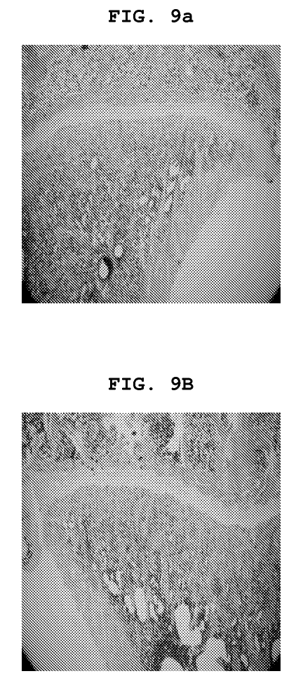

As a result of histological studies, the control showed a decrease in trabecular bone density in the bone marrow cavity and an elevated porosity in cortical bone. In contrast, the test group displayed a decrease in trabecular bone density and porosity in cortical bone (FIGS. 9a and 9b). These results indicate that the Compound 1 of the present invention has an excellent preventive effect on osteoporosis.

### 3-6. Therapeutic Effect of the Compound 1 on Osteoporosis

After ovariectomy, 20 female white mice were bred with solid foodstuff for four weeks according to the same method as described above. After checking the onset of osteoporosis in the mice, the mice were divided into a control group (4 mice) and a test group (16 mice) and orally administered with 220 μl of the Compound 1 dissolved in 30 ml water for four weeks. After checking change in body weight of the mice, tibia tissue specimens were fixed, dehydrated, cleared and stained for histological studies, according to a method known in the art.

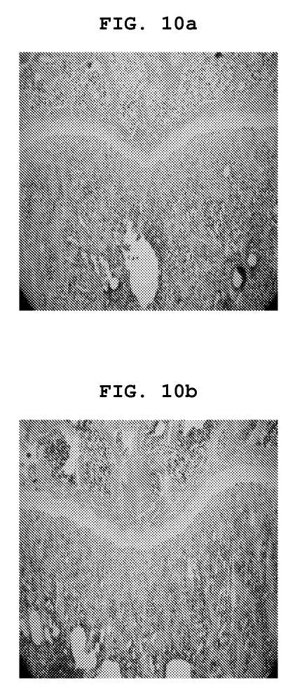

As a result of histological studies, a severe decrease in trabecular bone density was found in the control, whereas there was just a slight decrease in trabecular bone density in the test group (FIGS. 10a and 10b). These results indicate that the Compound 1 of the present invention has a therapeutic effect on advanced osteoporosis and thus an effect of preventing bone density reduction.

### Experimental Example 4

### Clinical Test Using Bone Mineral Densitometer

### 4-1. Preventive Effect of the Compound 1 on Osteoporosis

After ovariectomy, female white mice weighing about 250 g were orally administered with 10 mg of the Compound 1 per mice for four weeks. Bone mineral density (BMD) was measured before the ovariectomy and every week during the administration of the Compound 1, using a bone mineral densitometer, XCT 540 Research SA made in Germany.

After anesthetizing the mice being bred in cages by intraperitoneal injection of a mixture of Ketamine HCl (ketara 10 mg/kg) and 2% xylazine HCl (Roupun 0.15 ml/kg), BMD measurement was made at a voxel size of 0.1×0.1 mm² and threshold values of 280 mg/cm² for cancellous bone and 500 mg/cm² for compact bone. The analysis sites at the proximal tibias were determined by Scout scans (10 mm/sec), and BMD was measured at three slices at the determined sites by CT scans (7 mm/sec). BMD measurement was performed twice or more at the same site every week during four weeks.

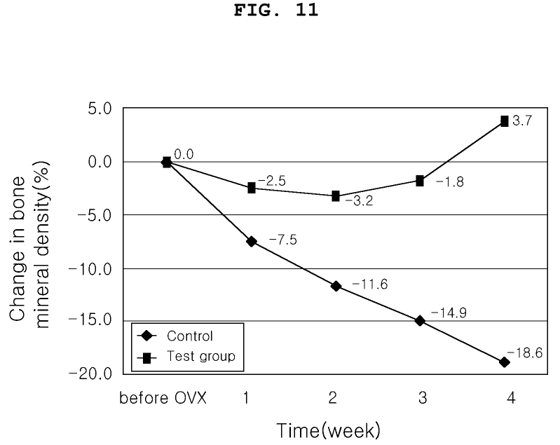

As a result, in the control group, BMD was decreased by about 19% 4 weeks after the ovariectomy. In contrast, the test group administered with the Compound 1 showed a similar BMD value during the administration period with the Compound 1 to that before the ovariectomy, and an increase in BMD after administration of the Compound 1 for four weeks, compared to the BMD value before the ovariectomy (Table 17 and FIG. 11). These results indicate that the Compound 1 of the present invention has an effect of preventing osteoporosis.

TABLE 17

Change in BMD, %

| Week | Control group | Test group |
|---|---|---|
| 0 | 0.0 | 0.0 |
| 1 | −7.5 | −2.5 |
| 2 | −11.6 | −3.2 |
| 3 | −14.9 | −1.8 |
| 4 | −18.6 | 3.7 |

### 4-2. Therapeutic Effect of the Compound 1 on Osteoporosis

After ovariectomy, female white mice weighing about 250 g were bred for four weeks. After checking the onset of osteoporosis in the mice, each of the mice was orally administered with 220 μl of the Compound 1 dissolved in 30 ml water for four weeks.

BMD measurement was performed before ovariectomy and every week for eight weeks after ovariectomy, using a bone mineral densitometer, XCT 540 Research SA made in Germany.

After anesthetizing the mice being bred in cages by intraperitoneal injection of a mixture of Ketamine HCl (ketara 10 mg/kg) and 2% xylazine HCl (Roupun 0.15 ml/kg), BMD measurement was made at a voxel size of 0.1×0.1 mm² and threshold values of 280 mg/cm² for cancellous bone and 500 mg/cm² for compact bone. The analysis sites at the proximal tibias were determined by Scout scans (10 mm/sec), and BMD was measured at three slices at the determined sites by CT scans (7 mm/sec). BMD measurement was performed twice or more at the same site every week during eight weeks after ovariectomy.

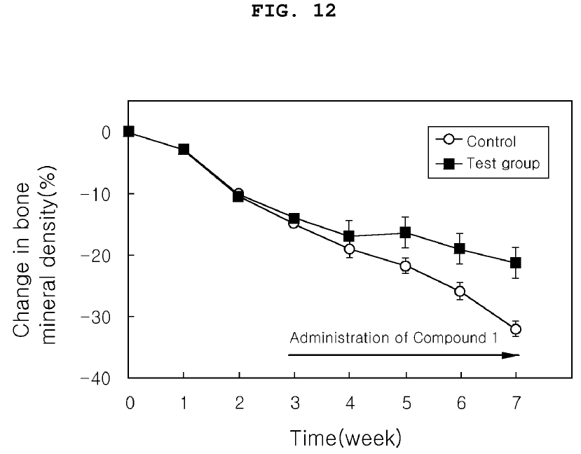

As a result, in a control group not administered with the Compound 1, BMD was decreased by about 22% after the ovariectomy. In contrast, in a test group administered with the Compound 1, BMD was decreased by about 15%, compared to that before the ovariectomy (Table 18 and FIG. 12). These results indicate that the Compound 1 of the present invention stimulates bone formation and thus has an effect of preventing osteoporosis.

TABLE 18

Change in BMD, %

| Week | Control group | Test group |
|---|---|---|
| 0 | 0 | 0 |
| 1 | −3 | −2.9 |
| 2 | −10 | −10.5 |
| 3 | −15 | −14 |
| 4 | −19 | −17 |
| 5 | −21.8 | −16.5 |
| 7 | −25.9 | −19 |
| 8 | −32 | −21.4 |
| 0 | 0 | 0 |

### Experimental Example 5

### Acute Oral Toxicity Test in Rats

The following experiments were performed to determine acute toxicity of compounds of this invention in rats.

6-week old SPF SD line rats were used in determining acute toxicity. The compounds of examples were suspended in 0.5% methylcellulose solution and orally administered once to 2 rats per group at the dosage of 1 g/kg/15 ml. Death, clinical symptoms, and weight change in rats were observed, hematological tests and biochemical tests of blood were performed, and any abnormal signs in the gastrointestinal organs of chest and abdomen were checked with eyes during autopsy.

The results showed that the test compounds did not cause any specific clinical symptoms, weight change, or death in rats. No change was observed in hematological tests, biochemical tests of blood, and autopsy. As a result, the compounds used in this experiment are evaluated to be safe substances since they do not cause any toxic change in rats up to the level of 3100 mg/kg and their estimated LD₅₀ values are much greater than 3100 mg/kg in rats.

### Preparation Example 1

### Preparation of Capsule

The compound of example 1 (5.0 mg) was mixed with 14.8 mg of lactose, 10.0 mg of polyvinyl pyrrolidone and 0.2 mg of magnesium stearate. The resultant mixture was filled with gelatine capsule.

The above capsule was comprising;

| | |
|---|---|
| The compound of example | 5.0 mg |
| Lactose | 14.8 mg |
| Polyvinyl pyrrolidone | 10.0 mg |
| Magnesium stearate | 0.2 mg |

### Preparation Example 2

### Preparation of Injectable Solution

The compound of example 1 (10 mg) was mixed with 180 mg of mannitol, 26 mg of Na₂HPO₄.12H₂O and 2974 mg of distilled water. The mixture was prepared to injectable solution. The injectable solution was sterilized at 20° C. for 30 min.

The above injectable solution was comprising;

| | |
|---|---|
| The compound of example 1 | 10 mg |
| Mannitol | 180 mg |
| Na₂HPO₄•12H₂O | 26 mg |
| Distilled water | 0.2 mg |

### Preparation Example 3

### Preparation of Beverage

The compound of example 1, vitamin C, powdery vitamin E, iron lactate, zinc oxide, nicotinic acid amide, Vitamin A, Vitamin B₁, Vitamin B₂ were mixed, to give beverage.

The above beverage was comprising;

| | |
|---|---|
| The compound of example 1 | 0.1 g |
| Vitamin C | 15 g |
| Powdery vitamin E | 7.5 g |
| Iron lactate | 19.75 g |
| Zinc oxide | 3.5 g |
| Nicotinic acid amide | 3.5 g |
| Vitamin A | 0.2 g |
| Vitamin B₁ | 0.25 g |
| Vitamin B₂ | 0.3 g |
| Distilled water | conditional-weight |

### INDUSTRIAL APPLICABILITY

As described hereinbefore, the furan derivatives of the present invention do not have problems encountered in the prior art, stimulate formation and activity of osteoblasts, as well as inhibiting formation and activity of osteoclasts. Therefore, the furan derivatives are useful for increasing children's height and preventing and treating osteoporosis, degenerative bone diseases, rheumatoid arthritis and other bone-related diseases. In addition, due to their non-toxicity, the furan derivatives are applicable as additives of health foods.

1-5. (canceled)

6. A method of preventing or treating bone disease comprising administering to a patient in need thereof a composition comprising a pharmaceutically effective amount of a compound represented by formula I or its pharmaceutically acceptable salt: 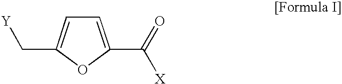
wherein, X represents H, OR, NR¹R², N—(CH₂CH₂)₂-A or N—(CH₂)ₙ;
Y represents OR, OCOR, NR¹R², N—(CH₂CH₂)₂-A, N—(CH₂)ₙ, OCONR¹R², ON═CR¹R², SC(NH₂)═NH or SR;
A represents CHCH₃, NCH₃ or O;
n is an integer from 4 to 6;
R represents hydrogen, naphthalene, aryl group having three or less substitution groups selected from among methyl, methoxy, chloro, bromo, iodo, nitro and fluorine, or a C₁-C₄ aliphatic alkyl group having four or less substituted fluorine; and
R¹ and R² are the same or different from each other and each represents hydrogen, naphthalene, aryl group having three or less substitution groups selected from among methyl, methoxy, chloro, bromo, iodo, nitro and fluorine, or a C₁-C₃ aliphatic alkyl group.

7. The method of claim 6, wherein the X and Y are selected from the group consisting of pairs of X and Y listed in the following Table 1:
TABLE 1

| No. | X | Y |
|---|---|---|
| 1 | H | HO— |
| 2 | H | CH3COO— |
| 3 | H | C6F5O— |
| 4 | H | CH3O— |
| 5 | H | 3,4-Cl2C6H3O— |
| 6 | H | 4-NO2C6H4O— |
| 7 | H | 2,4,6-Cl3C6H2O— |
| 8 | H | 4-BrC6H4O— |
| 9 | H | 3-CH3-4ClC6H3O— |
| 10 | H | C6Cl5O— |
| 11 | H | 4-CNC6H4O— |
| 12 | H | 3-CF3C6H4O— |
| 13 | H | 4-CH3OC6H4O— |
| 14 | H | 2,4-F2C6H3O— |
| 15 | H | 3-BrC6H4O— |
| 16 | H | 2-NO2C6H4O— |
| 17 | H | 2-BrC6H4O— |
| 18 | H | 3-Cl-4-FC6H3O— |
| 19 | H | 2-Cl-4-BrC6H3O— |
| 20 | H | 2-CH3OC6H4O— |
| 21 | H | 3-CH3-4-NO2C6H3O— |
| 22 | H | 2-Cl-4-FC6H3O— |
| 23 | H | 2,3-Cl2C6H3O— |
| 24 | H | 2-NO2-4-ClC6H3O— |
| 25 | H | 4-ClC6H4O— |
| 26 | H | 2,4-Cl2C6H3O— |
| 27 | H | 2-(CH3)2CH-4-Cl-5- |
| | | CH3C6H2O— |
| 28 | H | 2,4,6-Br3C6H2O— |
| 29 | H | 2-CH3C6H4O— |
| 30 | H | 2,6-(CH3)2C6H3O— |
| 31 | H | C6H5COO— |
| 32 | H | C6H5CH2COO— |
| 33 | H | 2,6-F2C6H3CH2COO— |
| 34 | H | 2-Cl-6-FC6H3CH2COO— |
| 35 | H | 3-Cl—C6H4CH2COO— |
| 36 | H | 3-SC4H3CH2COO— |
| 37 | H | 3-F—C6H4CH2COO— |
| 38 | H | 2-NpCH2COO— |
| 39 | H | 2,4-F2C6H3CH2COO— |
| 40 | H | (C6H5)3CCOO— |
| 41 | H | 2-CH3O-6-FC6H3CH2COO— |
| 42 | H | 3-CH3O-6-FC6H3CH2COO— |
| 43 | H | 2-BrCl4H28COO— |
| 44 | H | Cl4H29COO— |
| 45 | H | 4-FC6H4NHCOO— |
| 46 | H | C6H5NHCOO— |
| 47 | H | (CH3)2CHNHCOO— |
| 48 | H | 3-CF3C6H4NHCOO— |
| 49 | H | 3-ClC6H4NHCOO— |
| 50 | H | 4-BrC6H4NHCOO— |
| 51 | H | 2,4-(CH3O)2C6H3NHCOO— |
| 52 | H | C6H11NHCOO— |
| 53 | H | CH3CH2CH2CH2NHCOO— |
| 54 | H | 3,4-Cl2C6H3NHCOO— |
| 55 | H | 2-ClC6H4NHCOO— |
| 56 | H | CH3CH2NHCOO— |
| 57 | H | 2-NpNHCOO— |
| 58 | CH3O— | 3,5-Cl2-4- |
| | | NH2C6H2C(NH2)═NO— |
| 59 | CH3O— | 2-CH3O-4- |
| | | CH2═CHCH2C6H3O— |
| 60 | CH3O— | 2,4-Cl2C6H4O— |
| 61 | CH3O— | 2-ClC6H4O— |
| 62 | CH3O— | 2-BrC6H4O— |
| 63 | CH3O— | 2,4-Cl2C6H4O— |
| 64 | CH3O— | 2-NpO—* |
| 65 | CH3O— | C6F5O— |
| 66 | CH3O— | 2-NO2-4-ClC6H3O— |
| 67 | CH3O— | 2-NO2C6H4O— |
| 68 | CH3O— | 2-(CH3)2CH-5-CH3C6H3O— |
| 69 | CH3O— | 4-Cl—C6H4O— |
| 70 | CH3O— | 3,4-(CH2)3C6H3O— |
| 71 | CH3O— | 2-Cl-4-BrC6H3O— |
| 72 | CH3O— | 2-Cl-4-FC6H3O— |
| 73 | CH3O— | 3-CH3C6H4O— |
| 74 | CH3O— | 2-CH3-4-ClC6H3O— |
| 75 | CH3O— | 3-CH3-4-ClC6H3O— |
| 76 | CH3O— | 2,4-(CH3)C6H3O— |
| 77 | CH3O— | 3,5-(CH3)2-4-ClC6H2O— |
| 78 | CH3O— | 4-(CH3)2CHC6H4O— |
| 79 | CH3O— | 4-IC6H4O— |
| 80 | CH3O— | 4-ClC6H4O— |
| 81 | CH3O— | 3,4-(CH3)2C6H3O— |
| 82 | CH3O— | HN═C(NH2)S— |
| 83 | CH3O— | 2-NpO—* |
| 84 | CH3O— | C6F5O— |
| 85 | CH3O— | (CH3)2N— |
| 86 | CH3O— | HN═C(NH2)S— |
| 87 | CH3O— | (CH2)5N— |
| 88 | CH3O— | O(CH2CH2)2N— |
| 89 | CH3O— | C6H5NH— |
| 90 | CH3O— | (CH2)4N— |
| 91 | CH3O— | (CH3)3CNH— |
| 92 | CF3CH2O— | 2-NpO—* |
| 93 | (CH3)2CHO— | 4-(CH3)2CHC6H4O— |
| 94 | (CH3)2CHO— | 2-CH3OC6H4O— |
| 95 | (CH3)2CHO— | 2,5-Cl2C6H3O— |
| 96 | 2-ClC6H4O— | CH3CH2OC6H4O— |
| 97 | 4-ClC6H4O— | 2-CH3OC6H4O— |
| 98 | C6H5O— | 2-ClC6H4O— |
| 99 | CH2═CHCH2O— | 2,4-Cl2C6H3O— |
| 100 | HO— | 4-CH3CH2CH2C6H4O— |
| 101 | HO— | CHF2CF2CH2O— |
| 102 | HO— | 4-FC6H4O— |
| 103 | HO— | 4-BrC6H4O— |
| 104 | HO— | 2-NpO—* |
| 105 | HO— | 3-CF3C6H4C(CH3)═NO— |
| 106 | HO— | 2,4-Cl2C6H3O— |
| 107 | HO— | 2-ClC6H4O— |
| 108 | HO— | 2-BrC6H4O— |
| 109 | HO— | 2,5-Cl2C6H3O— |
| 110 | HO— | 4-FC6H4O— |
| 111 | HO— | 4-Cl-3-CH3C6H4O— |
| 112 | HO— | 3-ClC6H4O— |
| 113 | HO— | 2-NO2C6H4O— |
| 114 | HO— | 4-(CH3)2CHC6H4O— |
| 115 | HO— | 4-Cl-2-NO2C6H3O— |
| 116 | HO— | 3-CH3OC6H4O— |
| 117 | HO— | 1-NpO—* |
| 118 | HO— | 4-CH3CH2CH(CH3)C6H4O— |
| 119 | HO— | 4-Cl-3-CH3C6H3O— |
| 120 | HO— | C6H5CH2S- |
| 121 | CH3CH2CH(CH3)NH— | 4-IC6H4O— |
| 122 | 3-CH3C6H4NH— | 4-IC6H4O— |
| 123 | (CH3CH2)2N— | 4-Cl-3-CH3C6H3O— |
| 124 | CH3CH(CH2CH2)2N— | 2,5-Cl2C6H4O— |
| 125 | (CH2)4CHNH— | CF3CH2O— |
| 126 | (CH2)6CHNH— | 2-CH3OC6H4O— |
| 127 | (CH3)3CNH— | CF3CH2O— |
| 128 | (CH2)6N— | 2-BrC6H4O— |
| 129 | (CH3)3CNH— | 2-BrC6H4O— |
| 130 | (CH3)2CHNH— | 2,5-Cl2C6H3NH— |
| 131 | CH3N(CH2CH2)2N— | 2,5-Cl2C6H3O— |
| 132 | O(CH2CH2)2N— | 3-CH3-4-ClC6H3O— |
| 133 | (CH2)6N— | 2,5-Cl2C6H3O— |
| 134 | (CH2)5CHNH— | 4-(CH3)3CC6H4O— |
| 135 | (CH2)4NH— | 4-IC6H4O— |
| 136 | C6H5NH— | 4-(CH3)3CC6H4O— |
| 137 | C6H5NH— | 2-NpO—* |
| 138 | 4-ClC6H4NH— | 4-IC6H4O— |
| 139 | 3-F-4-CH3C6H3NH— | 4-IC6H4O— |
| 140 | 3-BrC6H4NH— | 4-FC6H4O— |
| 141 | 4-FC6H4NH— | 3-ClC6H4O— |
| 142 | 3-Cl-4-CH3OC6H3NH— | 2-ClC6H4O— |
| 143 | 3,4-F2C6H3NH— | 4-IC6H4O— |
| 144 | 2-CH3CH2OC6H4NH— | 2-NO2-4-ClC6H3O— |
| 145 | 2,4-(CH3O)2C6H3NH— | 2,5-Cl2C6H3O— |
| 146 | 4-BrC6H4NH— | 4-IC6H4O— |
| 147 | 4-FC6H4NH— | 2-NO2-4-CH3C6H3O— |
| 148 | 4-NH2COC6H4NH— | 2,5-Cl2C6H3O— |
| 149 | 2-NO2-4-CH3OC6H3NH— | 4-F—C6H4O— |
| 150 | 4-CH3OC6H4NH— | 3-CH3-4-ClC6H3O— |
| 151 | 2,5-F2C6H3NH— | 3-ClC6H4O— |
| 152 | 2-CH3-5-CH3O2CC6H3NH— | 4-IC6H4O— |
| 153 | 2-CH3O2CC6H4NH— | 2,5-(CH3)2C6H3O— |
| 154 | 3,5-(CH3)2C6H3NH— | 4-F—C6H4O— |
| 155 | 2-F-5-CH3C6H3NH— | 2-NO2-4-CH3C6H3O— |
| 156 | 2,3-Cl2C6H3NH— | 2-NO2C6H4O— |
| 157 | 2-CH3OC6H4NH— | 2,5-(CH3)2C6H3O— |
| 158 | 2-F-5-CH3C6H3NH— | 2-NO2C6H4O— |
| 159 | 2-CH3O2CC6H4NH— | 4-I—C6H4O— |
| 160 | 4-CH3COC6H4NH— | 1-NpO—* |
| 161 | 2,5-F2C6H3NH— | 2-ClC6H4O— |
| 162 | 2-F-4-BrC6H3NH— | 4-IC6H4O— |
| 163 | 3-CH3CH2C6H4NH— | 4-F—C6H4O— |
| 164 | 3,4-(CH3O)2C6H3NH— | 4-CH3CH2CH2C6H4O— |
| 165 | (CH3)3CNH— | 4-CH3CH2OC6H4O— |
| 166 | 2-CH3OC6H4NH— | 2-CH3OC6H4O— |
| 167 | 2-CH3O2CC6H4NH— | 2,4-Cl2C6H3O— |
| 168 | 2-CH3O2CC6H4NH— | 4-ClC6H4O— |
| 169 | 3,5-(CH3)2C6H3NH— | 4-CH3CH2—C6H4O— |
| 170 | 2,5-Cl2C6H3NH— | 4-CH3CH2CH2C6H4O— |
| 171 | 2-CH3O-5-CH3C6H3NH— | 3-ClC6H4O— |
| 172 | 2,3-(CH3)2C6H3NH— | 3-CH3-4-ClC6H3O— |
| 173 | 4-ClC6H4NH— | 3-CH3-4-ClC6H4O— |
| 174 | 2-ClC6H4NH— | 2-CH3OC6H4O— |
| 175 | 3,4-(CH3)2C6H4NH— | 2-CH3OC6H4O— |
| 176 | 2,4-F2C6H3NH— | 2-CH3O-4- |
| | | CH3CH2CH2C6H3O— |
| 177 | 2-FC6H4NH— | 3,5-(CH3)2C6H3O— |
| 178 | 2-FC6H4NH— | 4-ClC6H4O— |
| 179 | 2,6-(CH3)2C6H3NH— | 4-ClC6H4O— |
| 180 | 2-CH3O2CC6H4NH— | 3,4-(CH3)2C6H3O— |
| 181 | 2-Cl-5-CF3C6H3NH— | 4-ClC6H4O— |
| 182 | 2-CH3O-4-NO2C6H3NH— | 4-ClC6H4O— |
| 183 | 2-NO2C6H4NH— | 2-ClC6H4O— |
| 184 | 4-CH3OC6H4NH— | CHF2CF2CH2O— |
| 185 | 2,4-F2C6H3NH— | 2,3,5,6-F4C6HO— |
| 186 | 3,4-F2C6H3NH— | 3,4-(CH3)2C6H3O— |
| 187 | 3-CH3CONHC6H4NH— | C6F5O— |
| 188 | 2,4-(CH3O)2C6H4NH— | 2-BrC6H4O— |
| 189 | 2-CH3OC6H4NH— | 4-(CH3)3CC6H4O— |
| 190 | 4-IC6H4NH— | 2-BrC6H4O— |
| 191 | 3-NO2C6H4NH— | 4-IC6H4O— |

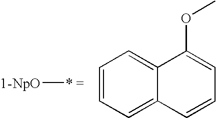

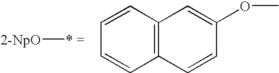

8. The method of claim 7, wherein the X is hydrogen and the Y is hydroxy.

9. The method of any one of claims 6 to 8, wherein said composition is in a capsule or injectable solution form.

10. A method of preventing bone disease comprising ingesting a food comprising an effective amount of the compound represented by formula I of claim 6 or its pharmaceutically acceptable salt.

11. The method of claim 10, wherein said food is in a beverage form.

12. The method of claim 6 or 10, wherein the bone disease is selected from the group consisting of osteoporosis, degenerative bone disease and rheumatoid arthritis.
